(19) United States
(12) Patent Application Publication
(10) Pub. No.: US 2011/0245846 A1
(43) Pub. Date: Oct. 6, 2011

(54) ENDOSCOPIC TISSUE ANCHOR DEPLOYMENT DEVICES AND METHODS

(75) Inventors: Richard C. EWERS, Fullerton, CA (US); Eugene G. CHEN, Carlsbad, CA (US); Tracy D. MAAHS, Yorba Linda, CA (US); John A. COX, San Clemente, CA (US)

(73) Assignee: USGI Medical, Inc., San Clemente, CA (US)

(21) Appl. No.: 13/034,448

(22) Filed: Feb. 24, 2011

Related U.S. Application Data
(60) Provisional application No. 61/307,764, filed on Feb. 24, 2010.

(51) Int. Cl. A61B 17/10

(52) U.S. Cl. 606/139

(57) ABSTRACT

An endoscopic tissue anchor deployment device includes a handle, an elongated shaft defining an internal lumen, and an end effector attached to the distal end of the elongated shaft. A tissue anchor catheter is removably inserted through the lumen of the elongated shaft, the catheter having a tissue anchor assembly that is deployable from its distal end. In some embodiments, the handle includes a pin and track assembly that defines a series of handle actuation steps corresponding to deployment steps for the deployment device end effector and the tissue anchor catheter. In some embodiments, the handle includes a catheter stop member that prevents movement of the tissue anchor catheter under certain circumstances, and a handle stop member that prevents actuation of the handle under certain circumstances.

### RELATED APPLICATION DATA

This application claims the benefit of priority to U.S. Provisional Patent Application No. 61/307,764, filed on Feb. 24, 2010, the contents of which are incorporated herein by reference in their entirety. This application also relates to the subject matter disclosed in the following U.S. Patent Application Ser. No. 61/432,537, filed Jan. 13, 2011, Ser. No. 12/486,578, filed on Jun. 17, 2009, Ser. No. 12/409,335, filed on Mar. 23, 2009, Ser. No. 61/239,709, filed on Sep. 3, 2009, Ser. No. 11/070,845, filed on Mar. 1, 2005, Ser. No. 11/002,369, filed on Dec. 1, 2004, and Ser. No. 10/955,245, filed on Sep. 29, 2004 (now U.S. Pat. No. 7,347,863). The contents of each of the foregoing patent applications are incorporated herein by reference in its entirety.

### BACKGROUND

The present disclosure relates to methods and apparatus for manipulating and/or securing tissue. More particularly, the present disclosure relates to methods and apparatus for manipulating and/or securing tissue endoscopically and/or endolumenally, for instance, to form tissue folds, to approximate regions of tissue, and/or to deploy tissue anchors.

A number of surgical techniques have been developed to treat various gastrointestinal disorders. One example of a pervasive disorder is morbid obesity. Conventional surgical treatment for morbid obesity typically includes, e.g., bypassing an absorptive surface of the small intestine, or reducing the stomach size. However, many conventional surgical procedures may present numerous life-threatening post-operative complications, and may cause atypical diarrhea, electrolytic imbalance, unpredictable weight loss and reflux of nutritious chyme proximal to the site of the anastomosis.

Furthermore, the sutures or staples that are often used in surgical procedures for gastrointestinal disorders typically require extensive training by the clinician to achieve competent use, and may concentrate significant force over a small surface area of the tissue, thereby potentially causing the suture or staple to tear through the tissue. Many of the surgical procedures require regions of tissue within the body to be approximated towards one another and reliably secured. The gastrointestinal lumen, for instance, includes four tissue layers, where the mucosa layer is the inner-most tissue layer followed by connective tissue, the muscularis layer, and where the serosa layer is the outer-most tissue layer.

One problem with conventional gastrointestinal reduction systems is that the anchors (or staples) should engage at least the muscularis tissue layer in order to provide a proper foundation. In other words, the mucosa and connective tissue layers typically are not strong enough to sustain the tensile loads imposed by normal movement of the stomach wall during ingestion and processing of food. In particular, these layers tend to stretch elastically rather than firmly hold the anchors (or staples) in position, and accordingly, the more rigid muscularis and/or serosa layer should ideally be engaged. This problem of capturing the muscularis or serosa layers becomes particularly acute where it is desired to place an anchor or other apparatus transesophageally rather than intra-operatively, since care must be taken in piercing the tough stomach wall not to inadvertently puncture adjacent tissue or organs.

One conventional method for securing anchors within a body lumen to the tissue is to utilize sewing devices to suture the stomach wall into folds. This procedure typically involves advancing a sewing instrument through the working channel of an endoscope and into the stomach and against the stomach wall tissue. The contacted tissue is then typically drawn into the sewing instrument where one or more sutures or tags are implanted to hold the suctioned tissue in a folded condition known as a plication. Another method involves manually creating sutures for securing the plication.

One of the problems associated with these types of procedures is the time and number of intubations needed to perform the various procedures endoscopically. Another problem is the time required to complete a plication from the surrounding tissue with the body lumen. In the period of time that a patient is anesthetized, procedures such as for the treatment of morbid obesity, revision of obesity procedures, or for GERD must be performed to completion. Accordingly, the placement and securement of the tissue plication should ideally be relatively quick and performed with a high degree of confidence.

Another problem with conventional methods involves ensuring that the staple, knotted suture, or clip is secured tightly against the tissue and that the newly created plication will not relax under any slack which may be created by slipping staples, knots, or clips. Other conventional tissue securement devices such as suture anchors, twist ties, crimps, etc. are also often used to prevent sutures from slipping through tissue. However, many of these types of devices are typically large and unsuitable for low-profile delivery through the body, e.g., transesophageally.

Moreover, when grasping or clamping onto or upon the layers of tissue with conventional anchors, sutures, staples, clips, etc., many of these devices are configured to be placed only after the tissue has been plicated and not during the actual plication procedure.

### SUMMARY

In one general aspect, devices according to the present invention include mechanisms for deploying tissue anchors and tissue anchor assemblies into and/or through tissue within a patient. In some embodiments, the devices are introduced endolumenally (e.g., transorally, transanally, etc.) into the patient's body and into or around the gastrointestinal (“GI”) tract. Once the instruments are positioned within the stomach or other target site, tissue at the target site is temporarily engaged or grasped and the engaged tissue is manipulated by a surgeon or practitioner from outside the patient's body.

In engaging, manipulating, and/or securing the tissue, various methods and devices may be implemented. For instance, tissue securement devices may be delivered and positioned via an endoscopic apparatus for contacting a tissue wall, creating one or more tissue folds, and deploying one or more tissue anchors through the tissue fold(s). The tissue anchor(s) may be disposed through the muscularis and/or serosa layers of the tissue. An endoscopic access device having an elongate body, a steerable distal portion, and multiple lumens defined therethrough may be advanced into a patient per-orally and through the esophagus. A tissue manipulation assembly positioned at the distal end of a tubular body may be passed through the endoscopic access device for engaging and securing the tissue.

Utilizing one or more of the instruments, the endoscopic access device may be used to pass the flexible body therethrough and into the stomach where it may be used to engage tissue and form folds, invaginations, or other reconfigurations of tissue which are secured via expandable tissue anchors expelled from the tissue manipulation assembly. Any number of tissue folds and/or invaginations, i.e., one or more, may be created.

In an embodiment, a delivery catheter is advanced through a patient's mouth and esophagus and into the patient's stomach or other target site, with the delivery catheter including a flexible tube having a needle at its distal end and with a first tissue anchor assembly being contained within the flexible tube of the delivery catheter. One or more instruments associated with the delivery catheter are used to form a first tissue fold in the tissue at the target site, the tissue fold preferably including a serosa-to-serosa contact of tissue on the peritoneal surface of the tissue. The needle of the delivery catheter is passed through the first tissue fold, and a first tissue anchor assembly is deployed from the delivery catheter through the first tissue fold to thereby secure the first tissue fold. A plurality of additional tissue folds may be also secured in the tissue.

In some embodiments, the devices and systems include an endoscopic access device, a tissue manipulation assembly, and a needle deployment assembly containing a tissue anchor or tissue anchor assembly. The tissue manipulation assembly includes a handle having several optional features and functions, including a handle stop member for preventing actuation of the handle under certain circumstances, a needle stop member for preventing advancement of the needle deployment assembly under certain circumstances, a pin and track mechanism that defines a series of handle actuation steps corresponding to deployment steps for the tissue manipulation assembly and the needle deployment assembly, and others.

### BRIEF DESCRIPTION OF THE DRAWINGS

FIG. 1A is a schematic representation of a tissue anchor assembly.

FIG. 1B is a schematic representation of a tissue anchor assembly securing a tissue fold.

FIG. 2A is a perspective view of a cinch.

FIG. 2B is a cross-sectional view of the cinch of FIG. 2A.

FIG. 3A is a side view of a tissue anchor delivery device, including a tissue manipulation assembly.

FIGS. 3B and 3C show detail side and perspective views, respectively, of the tissue manipulation assembly of the device of FIG. 3A

FIG. 4 is a side view of a needle deployment assembly which may be loaded or advanced into a tissue manipulation assembly.

FIG. 5 is a partial cross-sectional view of one variation of a needle and anchor assembly positioned within a launch tube.

FIG. 6A shows a tissue plication apparatus for creating tissue plications and delivering anchors into tissue, which may be detachably connected to an endoscope.

FIGS. 6B and 6C show the tissue plication apparatus of FIG. 6A detached from an endoscope and attached to the endoscope, respectively.

FIG. 7 is a side view of another embodiment of a tissue anchor delivery device, including a tissue manipulation assembly and a needle deployment assembly.

FIG. 8 is a side view of the tissue manipulation assembly of FIG. 7.

FIGS. 9A and 9B are perspective views of an end effector of the tissue manipulation assembly of FIGS. 7 and 8.

FIG. 10 is an exploded view of the needle deployment assembly of FIG. 7.

FIG. 11 is a side view of a tissue manipulation assembly extending from the distal end of a lumen of an endoscopic access device and deploying a tissue anchor assembly through a tissue fold.

FIGS. 12A and 12B are perspective views of endoscopic access devices.

FIG. 13 is a perspective view of an actuator mechanism for a tissue anchor delivery device.

FIGS. 14A-B are perspective views of another embodiment of a needle deployment assembly.

FIG. 15 is a side view of another embodiment of an actuator mechanism for a tissue anchor delivery device.

FIGS. 16A-B are perspective views of the actuator mechanism of FIG. 15.

FIG. 17 is a side view of an embodiment of a distal portion of a launch tube of a tissue anchor delivery device.

FIGS. 18A-C are a bottom view, side view, and expanded bottom view, respectively, of another embodiment of a distal portion of a launch tube of a tissue anchor delivery device.

FIGS. 19A-C are a top view, side view, and expanded side view, respectively, of another embodiment of a distal portion of a launch tube of a tissue anchor delivery device.

FIGS. 20A-B are a bottom view and expanded bottom view, respectively, of another embodiment of a distal portion of a launch tube of a tissue anchor delivery device.

FIGS. 21A-D are perspective views of an end effector of a tissue anchor delivery device.

FIGS. 22 and 22A are perspective views of an embodiment of a tissue anchor delivery device.

FIGS. 23 and 23A are perspective views of another embodiment of a tissue anchor delivery device.

FIGS. 24 and 24A are perspective views of another embodiment of a tissue anchor delivery device.

FIGS. 25 and 25A are perspective views of another embodiment of a tissue anchor delivery device.

FIG. 26 is a perspective view of the distal end of the device shown in FIGS. 23 and 23A, shown in retroflex position.

FIGS. 27A-I are schematic illustrations showing the progression of a tissue anchor deployment method.

FIGS. 28A-C are illustrations of another embodiment of a tissue anchor delivery device.

### DETAILED DESCRIPTION

Endoscopic and endolumenal surgical methods and devices are described herein. In several embodiments, the methods entail performing surgery through a patient's mouth or other natural orifices, reducing or eliminating the need for external incisions into the body. Operating through the body's natural orifices offers promise for faster healing times, less scarring and less pain which could lead to reduced hospitalization and quicker recovery.

USGI Medical, Inc. of San Clemente, Calif. has developed several devices and methods that facilitate endoscopic and endolumenal diagnostic and therapeutic procedures. Several endoscopic access devices are described, for example, in the following United States patent applications:

TABLE 1

| U.S. patent application Ser. No. | Filing Date |
|---|---|
| 10/346,709 | Jan. 15, 2003 |
| 10/458,060 | Jun. 9, 2003 |
| 10/797,485 | Mar. 9, 2004 |
| 11/129,513 | May 13, 2005 |
| 11/365,088 | Feb. 28, 2006 |
| 11/738,297 | Apr. 20, 2007 |
| 11/750,986 | May 18, 2007 |
| 12/061,591 | Apr. 2, 2008 |

Several tissue manipulation and tissue anchor delivery devices are described in the following United States patent applications:

TABLE 2

| U.S. patent application Ser. No. | Filing Date |
|---|---|
| 10/612,109 | Jul. 1, 2003 |
| 10/639,162 | Aug. 11, 2003 |
| 10/672,375 | Sep. 26, 2003 |
| 10/734,547 | Dec. 12, 2003 |
| 10/734,562 | Dec. 12, 2003 |
| 10/735,030 | Dec. 12, 2003 |
| 10/840,950 | May 7, 2004 |
| 10/955,245 | Sep. 29, 2004 |
| 11/070,863 | Mar. 1, 2005 |
| 12/486,578 | Jun. 17, 2009 |

Endolumenal tissue grasping devices are described in several of the United States patent applications listed above, and in the following United States patent applications:

TABLE 3

| U.S. patent application Ser. No. | Filing Date |
|---|---|
| 11/736,539 | Apr. 17, 2007 |
| 11/736,541 | Apr. 17, 2007 |

Tissue anchors are described in several of the United States patent applications listed above, and in the following United States patent applications:

TABLE 4

| U.S. patent application Ser. No. | Filing Date |
|---|---|
| 10/841,411 | May 7, 2004 |
| 11/404,423 | Apr. 14, 2006 |
| 11/773,933 | Jul. 5, 2007 |

Each of the foregoing patent applications is hereby incorporated by reference in its entirety.

Several endoscopic and/or endolumenal therapeutic procedures described in the above patent applications include the steps of acquiring (e.g., grasping) tissue to form a tissue fold and deploying or implanting a fold retaining device (e.g., a tissue anchor assembly) that is used to maintain the fold. Other such procedures include the steps of grasping at least two sections of tissue, approximating the at least two sections of tissue, and deploying or implanting a tissue retaining device (e.g., a tissue anchor assembly) that is used to maintain the at least two sections of tissue in their approximated state. For simplicity, the discussion herein will describe tissue anchor assemblies holding tissue folds, with it being understood that other portions or sections of tissue that do not constitute tissue folds are suitably retained by the tissue anchor assemblies. The following sections include descriptions of several embodiments of devices that are suitable for performing these and other endoscopic and/or endolumenal surgical procedures.

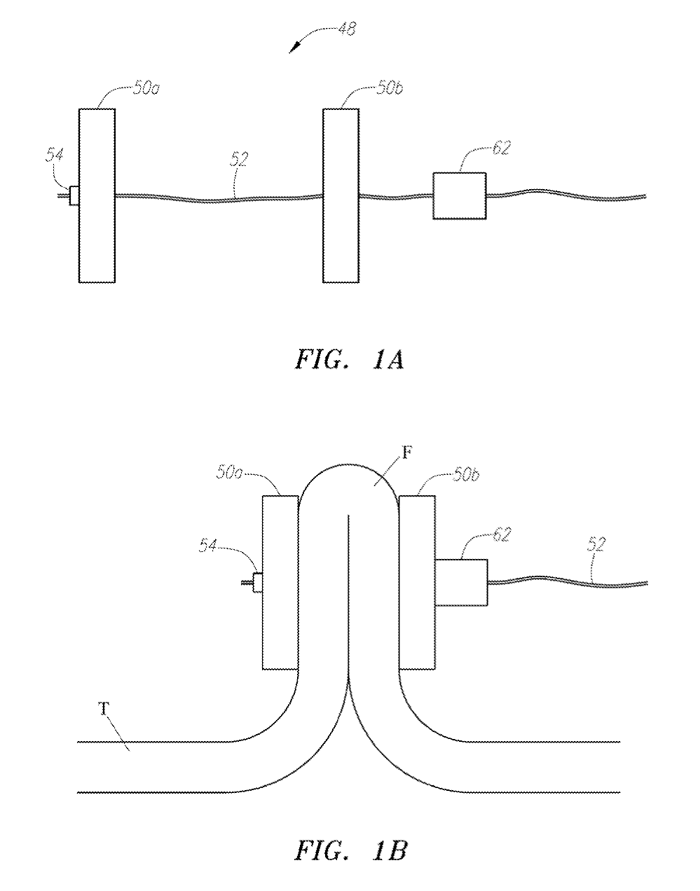

A tissue anchor assembly is used to maintain a tissue fold in tissue such as that present in the gastrointestinal lumen. Suitable tissue anchor assemblies include tissue anchors such as those described in several of the United States patent applications incorporated by reference above, including Ser. Nos. 10/841,411, 11/404,423, and 11/773,933. A schematic representation of a suitable tissue anchor assembly 48 is shown in FIG. 1A.

Preferably, the tissue anchor assemblies include a pair of tissue anchors 50a, 50b slidably retained by a connecting member, such as a suture 52. A locking mechanism, such as a cinch 62, is also slidably retained on the suture 52. The cinch 62 is configured to provide a cinching force against the anchors 50a, 50b in order to impart a tension force on the suture. Accordingly, the tissue anchor assembly 48 is adapted to hold a fold F of tissue T, as shown in FIG. 1B.

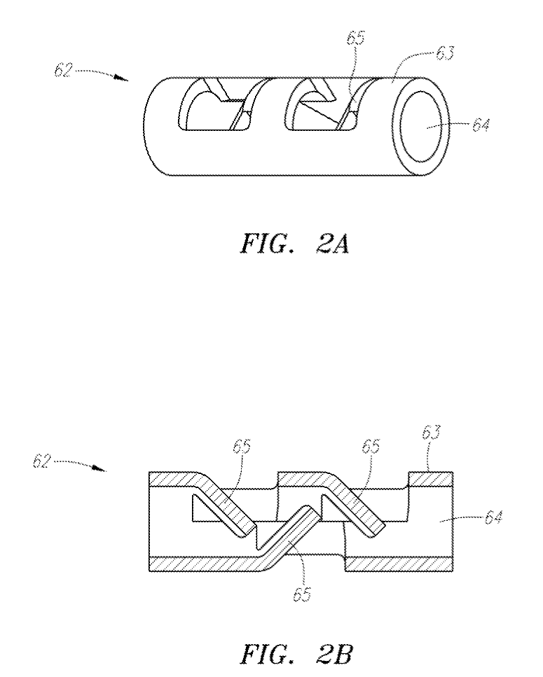

The cinch 62 functions by providing unidirectional translation over the suture thereby providing the ability to advance the tissue anchor(s) 50 into apposition and to retain the anchor(s) in place. An embodiment of a cinch 62 is shown in FIGS. 2A-B. The cinch includes a generally tubular body 63 defining an internal lumen 64. A plurality of inwardly facing levers 65 are formed integrally with the side wall of the tubular body 63. Three levers 65 are included in the cinch embodiment shown in the figures. In other embodiments, fewer levers (e.g., one or two) or more than three levers are used. In some embodiments, each lever 65 is flexibly biased to spring radially inward into the tubular body 63 or to deflect radially outward upon a suture 52 or other connector member passing therethrough. During translation of the suture 52 in a first direction (i.e., from left to right as viewed in FIG. 2B), the suture 52 is allowed to freely pass through the tubular body and past the plurality of levers due to a slight radially outward pivot of each of the levers. However, when the suture is urged in the second direction (i.e., from right to left as viewed in FIG. 2B), the levers 65 pivot radially inward, cinching down upon the suture against the inner surface of the tubular body 63. The cinching levers 65 are configured to prevent or inhibit the overcinching or cutting of the suture 52.

In other embodiments of the cinch 62, the levers 65 are substantially rigid, and do not pivot or deflect. In those embodiments, the levers 65 create a sufficiently tortuous path for the suture 52 (or other connector) to traverse that the cinch effectively binds the suture from translating in the first direction, while allowing translation in the second direction.

The cinches 62 described herein are formed of biocompatible and/or bioabsorbable materials such as those described above. In several embodiments, the cinch is formed of nickel-titanium alloy (Nitinol). The size and shape of the cinch are primarily dependent upon the size and shape of the other parts of the tissue anchor assembly, such as the diameter and materials forming the suture 52 (or other connector) and/or the size of the passage in the tissue anchors 50. Additional embodiments of cinches and additional cinching mechanisms suitable for use in the tissue anchor assemblies 48 are described and illustrated in U.S. patent application Ser. Nos. 10/612,170; 10/840,950; 10/840,951; 10/841,245; 10/841,411; 10/865,736; 11/036,866; 11/036,946; and 11/404,423, each of which is hereby incorporated by reference in its entirety (including all references cited therein) as if fully set forth herein.

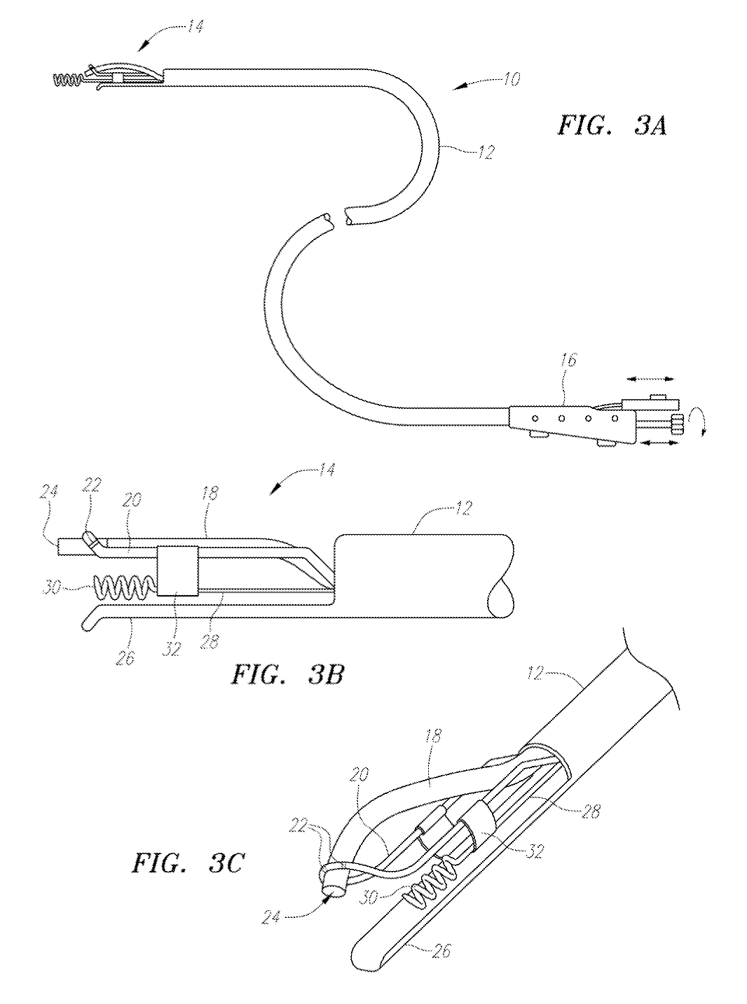

An illustrative side view of a tissue plication assembly 10 which may be utilized with the tissue anchors described herein is shown in FIG. 3A. The plication assembly 10 generally comprises a catheter or tubular body 12 which may be configured to be sufficiently flexible for advancement into a body lumen, e.g., transorally, percutaneously, laparoscopically, etc. The tubular body 12 may be configured to be torqueable through various methods, e.g., utilizing a braided tubular construction, such that when the handle 16 is manipulated and rotated by a practitioner from outside the body, the torquing force is transmitted along the body 12 such that the distal end of the body 12 is rotated in a corresponding manner.

A tissue manipulation assembly 14 is located at a distal end of the tubular body 12 and is generally used to contact and form the tissue plication, as mentioned above. FIG. 3B shows an illustrative detail side view and FIG. 3C shows a perspective view of the tissue manipulation assembly 14 which shows a launch tube 18 extending from the distal end of the body 12 and in-between the arms of an upper extension member or bail 20. The launch tube 18 defines a launch tube opening 24 and is pivotally connected near or at its distal end via a hinge or pivot 22 to the distal end of the upper bail 20. A lower extension member or bail 26 may similarly extend from the distal end of the body 12 in a longitudinal direction substantially parallel to the upper bail 20. The upper bail 20 and lower bail 26 need not be completely parallel so long as an open space between the upper bail 20 and lower bail 26 is sufficiently large enough to accommodate the drawing of several layers of tissue between the two members.

The upper bail 20 is shown in the figure as an open looped member and the lower bail 26 is shown as a solid member; however, this is intended to be merely illustrative and either or both members may be configured as looped or solid members. A tissue acquisition member 28 may be an elongate member, e.g., a wire, hypotube, etc., which terminates at a tissue grasper or engager 30, in this example a helically-shaped member, configured to be reversibly rotatable for advancement into the tissue for the purpose of grasping or acquiring a region of tissue to be formed into a plication. The tissue acquisition member 28 may extend distally from the handle 16 through the body 12 and distally between the upper bail 20 and the lower bail 26. The tissue acquisition member 28 may also be translatable and rotatable within the body 12 such that the tissue engager 30 is able to translate longitudinally between the upper bail 20 and the lower bail 26. To support the longitudinal and rotational movement of the acquisition member 28, an optional guide or linear bearing 32 may be connected to the upper 20 or lower bail 26 to freely slide thereon. A guide 32 may also be slidably connected to the acquisition member 28 such that the longitudinal motion of the acquisition member 28 is supported by the guide 32.

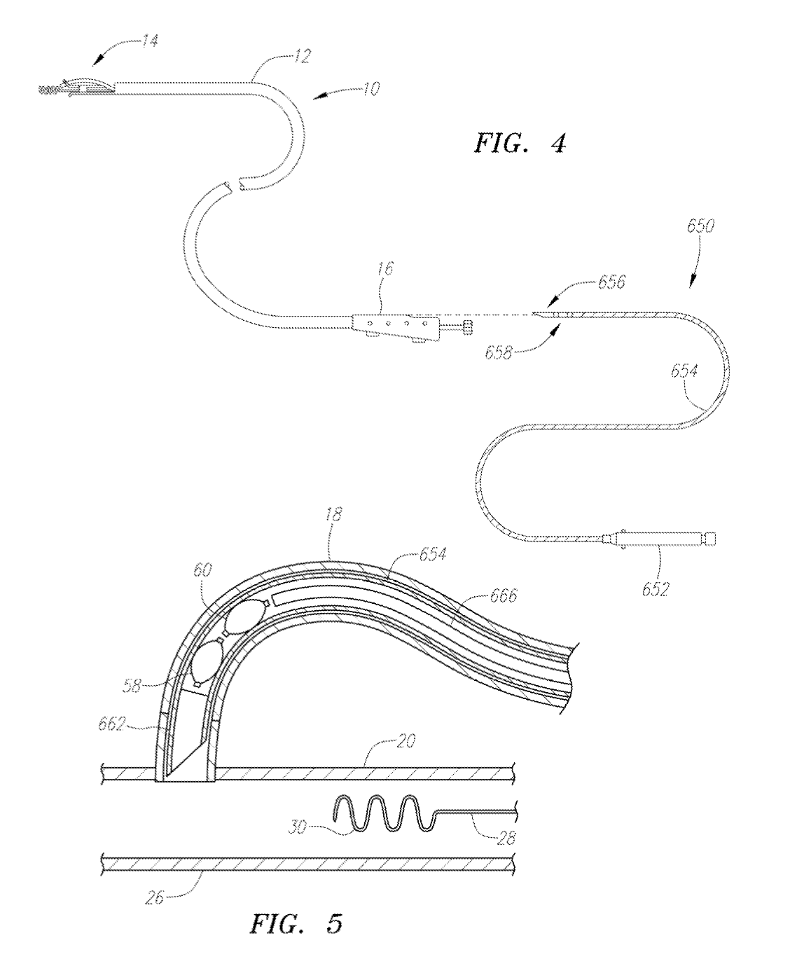

A needle deployment assembly 650 may be deployed through the tissue plication assembly 10 by introducing the needle deployment assembly 650 into the handle 16 and through the tubular body 12, as shown in the assembly view of FIG. 4, such that the needle assembly 656 is advanced from the launch tube and into or through approximated tissue. Once the needle assembly 656 has been advanced through the tissue, the anchor assembly 658 may be deployed or ejected. The anchor assembly 658 is normally positioned within the distal portion of the tubular sheath 654 which extends from the needle assembly control or housing 652. Once the anchor assembly 658 has been fully deployed from the sheath 654, the spent needle deployment assembly 650 may be removed from the tissue plication assembly 10 and another needle deployment assembly may be introduced without having to remove the assembly 10 from the patient. The length of the sheath 654 is such that it may be passed entirely through the length of the tubular body 12 to enable the deployment of the needle assembly 656 into and/or through the tissue.

FIG. 5 shows an illustrative cross-sectional view of the launch tube 18 in its deployment configuration. The tubular sheath 654 and needle body 662 of the needle deployment assembly 650 may be seen positioned within the distal portion of launch tube 18 ready for deployment into any tissue (not shown for clarity) which may be positioned between the upper and lower extension members 20, 26. Also shown are distal and proximal anchors 58, 60, respectively (the suture is not shown for clarity), positioned within the sheath 654 distally of the elongate pusher 666.

Additional details concerning the structure and function of the tissue plication assembly 10 and needle deployment assembly 650 shown in FIGS. 3A-C, 4, and 5 are contained in U.S. patent application Ser. No. 10/955,245, filed Sep. 29, 2004, (now U.S. Pat. No. 7,347,863), which is hereby incorporated by reference in its entirety.

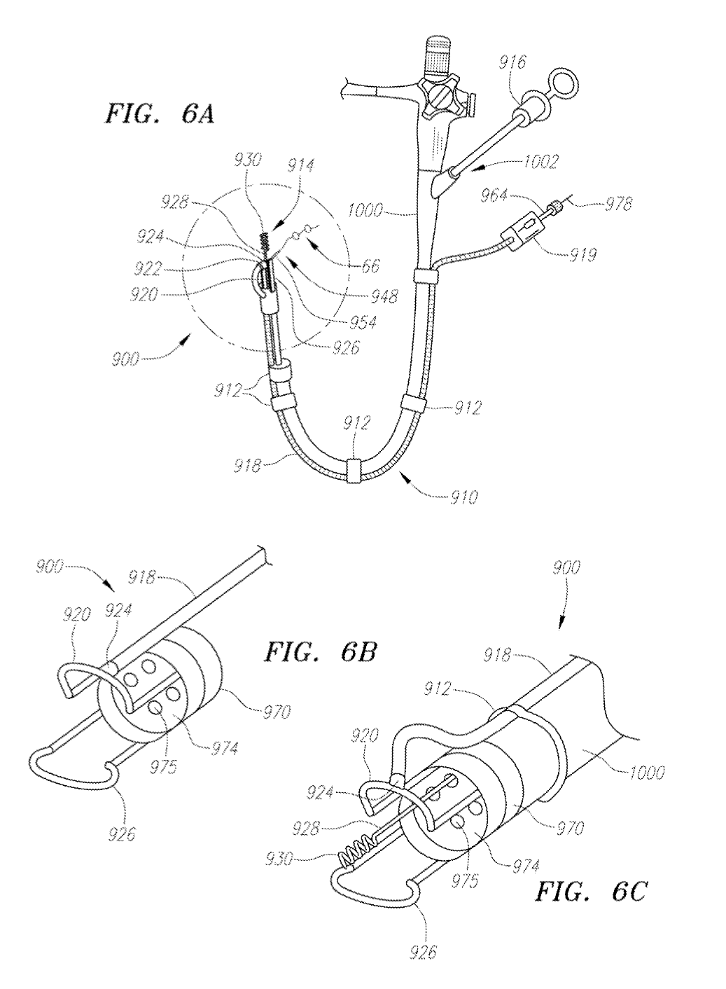

Turning next to FIGS. 6A-C, a variation of a tissue plication apparatus is described, which may be detachably connected to a standard endoscope. The tissue plication apparatus 900 generally comprises a tissue plication assembly 910, which is similar to the previously described tissue plication assembly 10, except that the assembly 910 does not comprise a catheter or tubular body 12. Rather, the assembly 910 comprises a tissue manipulation assembly 914, which is located at the distal end of a launch tube 918 and is generally used to contact tissue and form the tissue plication. One or more straps or other connectors 912 may be attached to the launch tube 918 along its length for reversibly securing the apparatus 900 to an endoscope 1000. The connectors 912 may, for example, comprise elastic bands or hook-and-loop, e.g., Velcro™ straps, etc. Alternatively, the connectors may comprise molded elements. Similar attachment mechanisms have been described previously, for example, in U.S. patent application Publication No. US2004/0147941 to Takemoto et al., which is incorporated herein by reference in its entirety.

The launch tube 918 extends from a proximal launch tube control 919 to the tissue manipulation assembly 914 and in-between the arms of an upper extension member or bail 920. The launch tube 918 defines a launch tube opening 924 and is pivotally connected near or at its distal end via a hinge or pivot 922 to the distal end of the upper bail 920. A lower extension member or bail 926 similarly extends distally in a longitudinal direction substantially parallel to the upper bail 920. The upper bail 920 and lower bail 926 need not be completely parallel so long as an open space between the upper bail 920 and lower bail 926 is sufficiently large enough to accommodate the drawing of up to several layers of tissue between the two members. The upper bail 920 and/or lower bail 926 may similarly be configured as practicable in any of the bail variations described herein or in the applications incorporated by reference herein.

A tissue acquisition member 928 may be an elongate member, e.g., a wire, hypotube, etc., or any of the variations as described herein, which terminates at a tissue grasper or engager 930, in this example a helically-shaped member configured to be reversibly rotatable for advancement into the tissue, for the purpose of grasping or acquiring a region of tissue to be formed into a plication. Alternatively, the tissue grasper or engager 930 may be formed in any of the tissue grasping variations as described herein. The tissue acquisition member 928 extends distally from the tissue acquisition control 916 through a working channel 1002 of the endoscope 1000 and distally between the upper bail 920 and lower bail 926. The tissue acquisition member 928 is translatable and rotatable within the working channel 1002 such that the tissue engager 930 is able to translate longitudinally and rotate between the upper bail 920 and lower bail 926. To support the longitudinal and rotational movement of the acquisition member 928, an optional guide or linear bearing (not shown) may be connected to the upper bail 920 or lower bail 926 to freely slide thereon. The guide may also be slidably connected to the tissue acquisition member 928, such that the longitudinal motion of the tissue acquisition member 928 is supported by the guide.

It is expected that reversibly attaching the tissue plication apparatus 900 to a standard endoscope 1000 will reduce a cross-sectional profile of the composite apparatus, as compared to providing an endoscope and tissue plication apparatus that do not attach to one another. The composite profile may be reduced to roughly that of the endoscope 1000. This is achieved by utilizing the working channel 1002 of the endoscope 1000 for advancement of the tissue acquisition member 928, and by obviating a need for a stand-alone catheter body for the apparatus 900. Furthermore, the endoscope 1000 may facilitate positioning of the apparatus 900 at a tissue site of interest by utilizing the steering capabilities of the endoscope 1000.

It is expected that providing a fixed distance between the distal end of the endoscope 1000 and the distal end of the assembly 914 may facilitate direct visualization via the endoscope 1000. Furthermore, a fixed distance may facilitate actuation of the launch tube 918, delivery of the needle 954 across the bails 920 and 926, and/or deployment of the anchor assemblies across tissue folds. Thus, tissue manipulation assembly 914 optionally may be reversibly coupled to a distal region of the endoscope 1000. For example, a proximal extension of the assembly may be positioned within a distal end of the endoscope working channel 1002, as described in more detail in U.S. application Ser. No. 11/002,369, filed Dec. 1, 2004, which is hereby incorporated by reference herein.

The launch tube 918 is typically configured to partially translate relative to the tissue manipulation assembly 914, e.g., via the launch tube control 919, such that a distal portion of the launch tube 918 may be articulated perpendicularly or transverse to tissue drawn between the bails 920 and 926. Thus, in this particular variation, at least a portion of the launch tube 918, or an actuator, preferably translates relative to the endoscope 1000. The connectors 912 may, for example, comprise through-holes or lumens through which the launch tube 918 is translationally disposed. In such a configuration, the connectors may, for example, comprise rigid connectors that are molded or machined, and then advanced over the endoscope 1000. The launch tube 918 then may be advanced through the lumens or through-holes of the connectors.

Alternatively or additionally, the launch tube 918 may comprise coaxially-disposed inner and outer tubes. The outer tube may be statically coupled to the connectors 912, and thereby the endoscope 1000, while the inner tube may be configured to translate relative to the outer tube. In this manner, the inner tube may partially translate relative to the tissue manipulation assembly 914. Additional methods and apparatus for translating the launch tube 918, per se known, will be apparent.

With the launch tube 918 articulated perpendicularly or transverse to tissue drawn between the bails 920 and 926, a needle 954 of a needle assembly 948 (e.g., previously described needle assembly 48) may be advanced through the lumen of the launch tube 918 via manipulation from its proximal end at the launch tube control 919 through a delivery push tube or catheter 964. The needle 954 preferably is a hollow needle having a tapered or sharpened distal end to facilitate its travel into and/or through tissue. The needle 954 may define a needle lumen through which, e.g., basket anchor assembly 66 may be situated during deployment and positioning of the assembly. An anchor push tube 978, disposed within the push tube 964 and needle assembly 948, may be used to deploy the basket anchor assembly 66 from the needle 954, as described above.

In FIG. 6A, although the tissue acquisition member 928 illustratively is shown advanced through the working channel 1002 of the endoscope 1000, it should be understood that the tissue acquisition member 928 alternatively may be advanced alongside of the endoscope 1000. Furthermore, the anchor launch tube 918 may alternatively or additionally be advanced through the working channel 1002, either alone or alongside the tissue acquisition member 928. Additionally, an endoscope with multiple working channels or lumens may be provided, and the launch tube 918 and tissue acquisition member 928 may be advanced through separate endoscope lumens. Advancing the launch tube 918 and/or tissue acquisition member 928 through one or more working channels of an endoscope may reversibly couple the apparatus 900 to the endoscope, thereby obviating a need for the connectors 912. Likewise, connection of the tissue manipulation assembly 914 to the distal region of the endoscope, e.g., via coupling to a working channel or a distal attachment, may also obviate a need for the connectors.

All, or a portion of, the apparatus 900 may be configured for single-use, i.e., may be disposable. Alternatively or additionally, all, or a portion of, the apparatus 900 may be configured for sterilization and re-use. The apparatus 900 optionally may be reversibly attached to alternative endoscopic or laparoscopic tools to achieve tissue folding.

FIG. 6B shows a perspective view of the apparatus 900 unattached to the endoscope 1000 for clarity. FIG. 6C shows a perspective view of the apparatus 900 having been attached to the distal end of the endoscope 1000 with the launch tube 918 and tissue grasper 930 deployed. The tissue grasper 930 and the tissue acquisition member 928 may be seen deployed and extending through an opening in the plate 974 from the working channel of the endoscope 1000. Several other openings, e.g., for suction, insufflation, visualization, etc., may be seen defined within the plate 974.

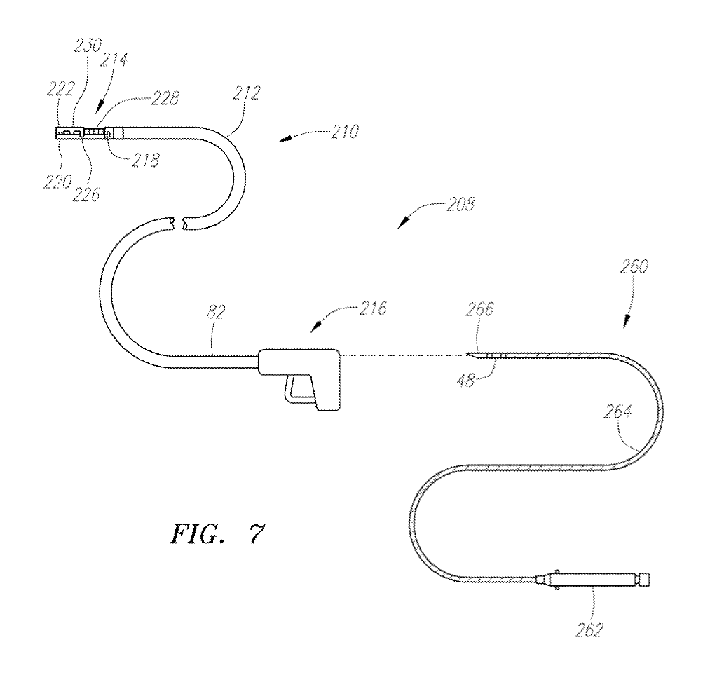

Turning next to the device embodiments shown in FIGS. 7-11, there is shown an example of a delivery device that is described in more detail in U.S. patent application Ser. No. 11/070,846, which is hereby incorporated by reference in its entirety (including all references cited therein) as if fully set forth herein. The delivery device 208 is described briefly below.

In manipulating tissue or creating tissue folds, a device having a distal end effector may be advanced endoscopically or endolumenally, e.g., transorally, transgastrically, etc., into the patient's body, e.g., the stomach. The tissue may be engaged or grasped and the engaged tissue may be manipulated by a surgeon or practitioner from outside the patient's body. Examples of creating and forming tissue plications are described in further detail in U.S. patent application Ser. No. 10/955,245, filed Sep. 29, 2004, which is incorporated herein by reference, as well as U.S. patent application Ser. No. 10/735,030, filed Dec. 12, 2003, which is also incorporated herein by reference in its entirety.

In engaging, manipulating, and/or securing the tissue, various methods and devices may be implemented. For instance, tissue securement devices may be delivered and positioned via an endoscopic apparatus for contacting a tissue wall of the gastrointestinal lumen, creating one or more tissue folds, and deploying one or more tissue anchors through the tissue fold(s). The tissue anchor(s) may be disposed through the muscularis and/or serosa layers of the gastrointestinal lumen.

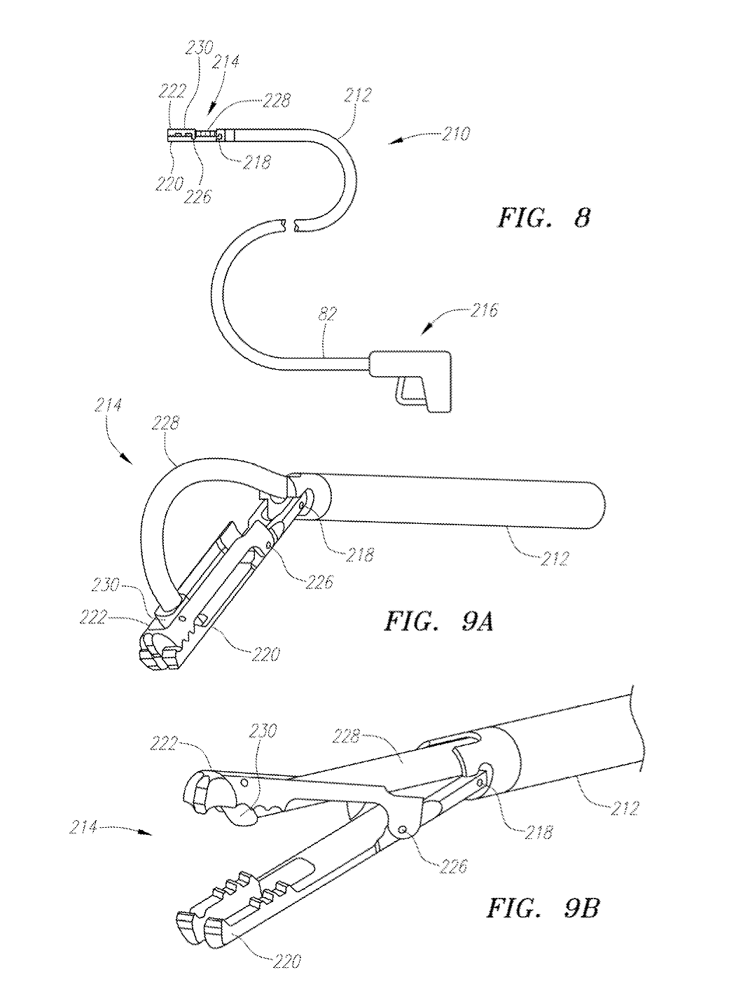

The delivery device 208 shown in FIG. 7 generally comprises a tissue manipulation assembly 210 and a needle deployment assembly 260. The tissue manipulation assembly 210 is also shown in FIG. 8. The tissue manipulation assembly 210 includes a flexible catheter or tubular body 212 which is configured to be sufficiently flexible for advancement into a body lumen, e.g., transorally, percutaneously, laparoscopically, etc. The tubular body 212 is configured to be torqueable through various methods, e.g., utilizing a braided tubular construction, such that when a handle 216 is manipulated and/or rotated by a practitioner from outside the patient's body, the longitudinal and/or torquing force is transmitted along the body 212 such that the distal end of the body 212 is advanced, withdrawn, or rotated in a corresponding manner.

For example, in some embodiments, the tubular body 212 has a composite construction that includes a first layer or multiple layers of braided wire or mesh and a second layer or multiple layers of a polymeric material such as polyurethane, nylon, polyester, Pebax (polyether block amide), or the like. An optional inner liner of polytetraflouroethylene (PTFE) may be provided to seal and/or to improve friction characteristics. The physical properties (e.g., hardness, stiffness) of a composite construction tubular body 212 will vary depending upon the materials used, the specific construction, and other factors.

In some embodiments, the tubular body 212 includes a proximal section having a first hardness and/or stiffness, and a distal section having a second hardness and/or stiffness that is lower than the hardness and/or stiffness of the proximal section. In these embodiments, the proximal section corresponds with a portion of the tubular body 212 that traverses a relatively non-tortuous path (e.g., through the relatively straight esophagus), and the distal section corresponds with a portion of the tubular body 212 that traverses a relatively tortuous path (e.g., bending regions of the tubular body or endoscopic access device that are guided or steered to reach portions of the stomach, colon, peritoneum, etc.). In this way, the tubular body 212 provides improved maneuverability during deployment, either as a standalone instrument or as deployed through a channel of an endoscopic access device. In particular, a distal section having a relatively lower hardness and/or stiffness will provide an improved capability to be rotated around its longitudinal axis within the channel of an endoscopic access device in comparison to a distal section having a relatively higher hardness and/or stiffness.

In a preferred embodiment, the proximal and distal sections are each formed as composite tubes including an inner liner of PTFE, a layer of stainless steel wire braid, and a layer of Pebax block copolymer. The proximal section includes a braid wire formed from round wire having a diameter of from about 0.004″ to about 0.008″ and having from about 30 picks per inch (“ppi”) to about 60 ppi, and a layer of Pebax having a Shore D hardness of from about 30 to about 45, and/or a flexural modulus (ASTM D 790) of from about 72 MPa to about 87 MPa. The distal section includes a braid wire formed from round wire having a diameter of from about 0.004″ to about 0.008″ and having from about 45 ppi to about 75 ppi, and a layer of Pebax having a Shore D hardness of from about 20 to about 35, and/or a flexural modulus (ASTM D 790) of from about 15 MPa to about 28 MPa. Those skilled in the art will recognize that other combinations of materials, material properties, and constructions of the tubular body 212 are possible. Moreover, those skilled in the art will recognize that sections additional to the proximal section and distal section may be added, with each section having different performance characteristics, in order to obtain desired performance.

A tissue manipulation end effector 214 is located at the distal end of the tubular body 212 and is generally used to contact and form tissue folds and/or to otherwise bring portions of tissue into apposition. The end effector is also shown in FIGS. 9A and 9B. The tissue manipulation end effector 214 is connected to the distal end of the tubular body 212 via a pivotable coupling 218. A lower jaw member 220 extends distally from the pivotable coupling 218 and an upper jaw member 222, in this example, is pivotably coupled to the lower jaw member 220 via a jaw pivot 226. The location of the jaw pivot 226 may be positioned at various locations along the lower jaw 220 depending upon a number of factors, e.g., the desired size of the “bite” or opening for accepting tissue between the jaw members, the amount of closing force between the jaw members, etc. One or both jaw members 220, 222 may also have a number of protrusions, projections, grasping teeth, textured surfaces, etc. on the surface or surfaces of the jaw members 220, 222 facing one another to facilitate the adherence of tissue between the jaw members 220, 222.

A launch tube 228 extends from the handle 216, through the tubular body 212, and distally from the end of the tubular body 212 where a distal end of the launch tube 228 is pivotally connected to the upper jaw member 222 at a launch tube pivot 230. A distal portion of the launch tube 228 may be pivoted into position within a channel or groove defined in upper jaw member 222, to facilitate a low-profile configuration of tissue manipulation end effector 214. When articulated, either via the launch tube 228 or other mechanism, the jaw members 220, 222 may be urged into an open configuration to receive tissue in the opening between the jaw members 220, 222. (See, e.g., FIG. 9B).

The launch tube 228 may be advanced from its proximal end at the handle 216 such that the portion of the launch tube 228 that extends distally from the body 212 is forced to rotate at a hinge or pivot 230 and reconfigure itself such that the exposed portion forms a curved or arcuate shape that positions the launch tube opening perpendicularly relative to the upper jaw member 222. (See, e.g., FIG. 9A). The launch tube 228, or at least the exposed portion of the launch tube 228, may be fabricated from a highly flexible material or it may be fabricated, e.g., from Nitinol tubing material which is adapted to flex, e.g., via circumferential slots, to permit bending.

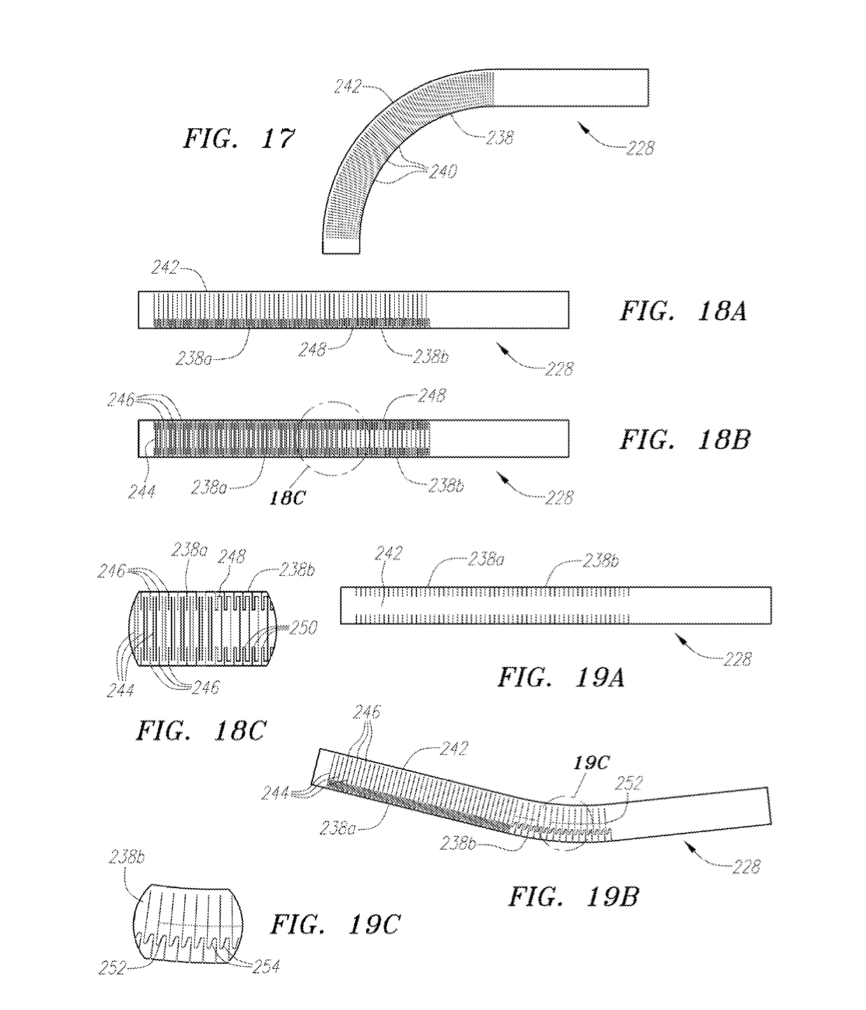

For example, turning to FIGS. 17, 18A-C, 19A-C, and 20A-B, four embodiments of a flexible distal portion 238 of a launch tube 228 are shown. In the first embodiment, shown in FIG. 17, a plurality of substantially uniformly spaced circumferential slots 240 are formed over the distal portion 238. In the embodiment, each of the circumferential slots 240 is of a substantially uniform length, with each extending radially around greater than 50% of the circumference of the launch tube. In some embodiments, the circumferential slots extend radially around the circumference of the launch tube by up to about 85% of the circumference and, in still other embodiments, the circumferential slots extend around the launch tube by up to about 90% of the circumference. A spine 242—i.e., the unslotted longitudinal section of the distal portion 238 of the launch tube—is defined along the length of the distal portion 238. In some embodiments, the spine has a width of between about 10% to about 15% or more of the circumference of the launch tube. As shown in FIG. 17, the size, shape, pattern, and orientation of the circumferential slots 240 and the spine 242 cause the distal portion 238 to flex and to effectively lock out at a predetermined position when a distally-oriented force is applied to the proximal end of the launch tube 228. The material, diameter, and wall thickness of the distal portion 238 of the launch tube also contribute to the degree of flex and predetermined lock out position. The predetermined lock-out position provides a relatively high degree of planar stability to the distal region 238 of the launch tube when it is in the flexed condition.

The launch tube distal region 238 embodiment shown in FIGS. 18A-C includes two regions 238a, 238b, each having a plurality of circumferential slots 240 formed in a pattern different from the other. In the first region 238a, located near the distal end of the distal portion 238, the circumferential slots include an alternating pattern including adjacent pairs of central semi-circumferential slots 244 centered upon the bending radius of the launch tube distal region 238. Each adjacent pair of central semi-circumferential slots 244 is separated by a pair of outer semi-circumferential slots 246. Each of the central slots 244 and outer slots 246 has a length of slightly less than 50% of the circumference of the distal region 238 of the launch tube. The resulting pattern is substantially in the form of a repetition of two longitudinally-aligned central slots 244 following by two radially aligned outer slots 246 along the length of the first region 238a. In the second region 238b, located just proximally of the first region 238a, the circumferential slots include a right-angle zigzag pattern 248 including nine adjoining segments each defining a right angle with its adjoining sections. The right-angle zigzag pattern 248 defines a plurality of longitudinally aligned keyed segments 250 on each side of the central bending radius of the launch tube distal region 238. The keyed segments 250 serve an additional function of substantially inhibiting over-extension in the reverse direction of the predetermined bending position, which may permanently deform or otherwise damage the distal region 238 of the launch tube.

Turning next to FIGS. 19A-C, the launch tube distal region 238 shown there includes a first region 238a having the identical circumferential slot pattern described above in relation to the first region 238a of the launch tube shown in FIGS. 18A-C, including central slots 244 and outer slots 246. The second region 238b includes slots defining a modified zigzag pattern 252 that includes at least two diagonal segments of the nine slot segments making up the modified zigzag pattern. The modified zigzag pattern 252 defines a plurality of longitudinally aligned keyed segments 254 on each side of the central bending radius of the launch tube distal region 238. The keyed segments 254 serve an additional function of substantially inhibiting over-extension in the reverse direction of the predetermined bending position, which may permanently deform or otherwise damage the distal region 238 of the launch tube.

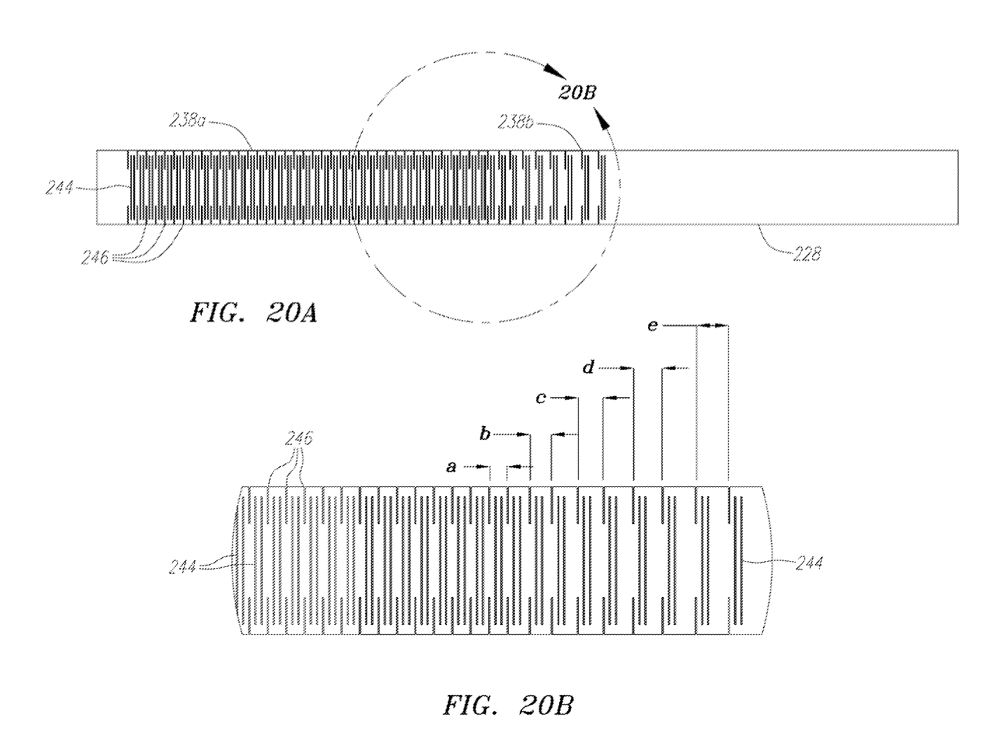

Finally, turning to FIGS. 20A-B, the launch tube distal region 238 shown there includes a first region 238a also having the identical circumferential slot pattern described above in relation to the first region 238a of the launch tube embodiments shown in FIGS. 18A-C and 19A-C, including central slots 244 and outer slots 246. The second region 238b also includes alternating central slots 244 and outer slots 246, but the alternating pattern includes slots that are spaced further apart longitudinally as the pattern progresses toward the proximal end. A representative example of the pattern spacing is shown in FIG. 20B, in which the relationships between the illustrated dimensions are a<b<c<d<e.

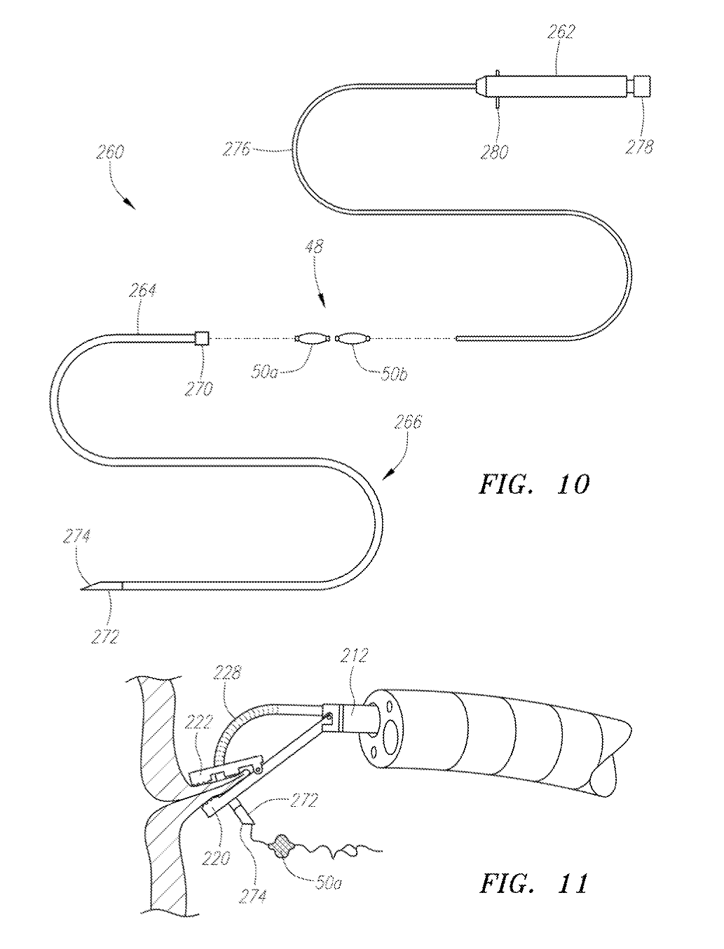

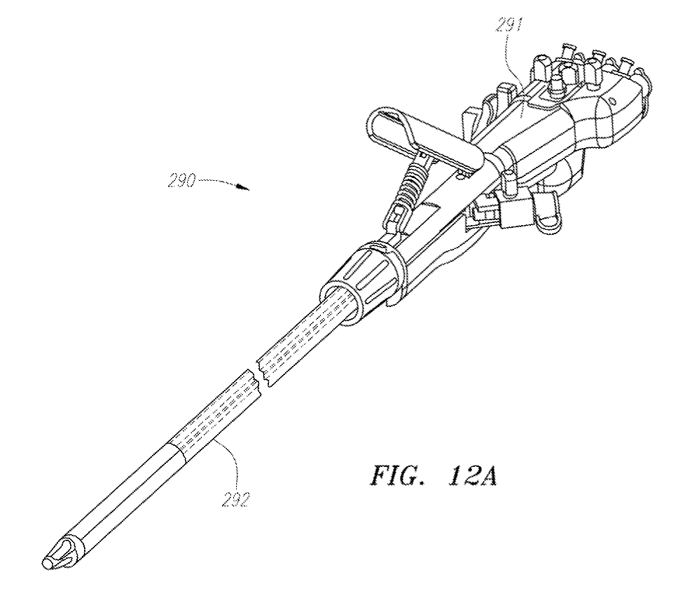

Returning again to FIGS. 7-11, once the tissue has been engaged between the jaw members 220, 222, a needle deployment assembly 260 is urged through the handle 216, though the tubular body 212, and out through the launch tube 228. The needle deployment assembly 260 may pass through the lower jaw member 220 via a needle assembly opening (not shown in the drawing) defined in the lower jaw member 220 to pierce through the grasped tissue. Once the needle deployment assembly has been passed through the engaged tissue, one or more tissue anchors of a tissue anchor assembly 48 (see FIG. 11) are deployed for securing the tissue, as described in further detail herein and in U.S. patent application Ser. No. 10/955,245, which has been incorporated by reference above.

FIG. 10 shows additional details relating to the needle deployment assembly 260. As mentioned above, a needle deployment assembly 260 may be deployed through the tissue manipulation assembly 210 by introducing needle deployment assembly 260 into the handle 216 and through the tubular body 212, as shown in the assembly view of FIG. 7, such that the needle assembly 266 is advanced from the launch tube and into or through approximated tissue. Once the needle assembly 266 has been advanced through the tissue, the anchor assembly 48 may be deployed or ejected. The anchor assembly 48 is normally positioned within the distal portion of a tubular sheath 264 that extends from a needle assembly control or housing 262. Once the anchor assembly 48 has been fully deployed from the sheath 264, the spent needle deployment assembly 260 may be removed from the tissue manipulation assembly 210 and another needle deployment assembly may be introduced without having to remove the tissue manipulation assembly 210 from the patient. The length of the sheath 264 is such that it may be passed entirely through the length of the tubular body 212 to enable the deployment of the needle assembly 266 into and/or through the tissue.

The elongate and flexible sheath or catheter 264 extends removably from the needle assembly control or housing 262. The sheath or catheter 264 and the housing 262 may be interconnected via an interlock 270 which may be adapted to allow for the securement as well as the rapid release of the sheath 264 from the housing 262 through any number of fastening methods, e.g., threaded connection, press-fit, releasable pin, etc. The needle body 272, which may be configured into any one of the variations described above, extends from the distal end of the sheath 264 while maintaining communication between the lumen of the sheath 264 and the needle opening 274.

An elongate pusher 276 comprises a flexible wire or hypotube that is translationally disposed within the sheath 264 and movably connected within the housing 262. A proximally-located actuation member 278 is rotatably or otherwise connected to the housing 262 to selectively actuate the translational movement of the elongate pusher 276 relative to the sheath 264 for deploying the anchors from the needle opening 274. The tissue anchor assembly 48 is positioned distally of the elongate pusher 276 within the sheath 264 for deployment from the sheath 264. Needle assembly guides 280 protrude from the housing 262 for guidance through the locking mechanism described above.

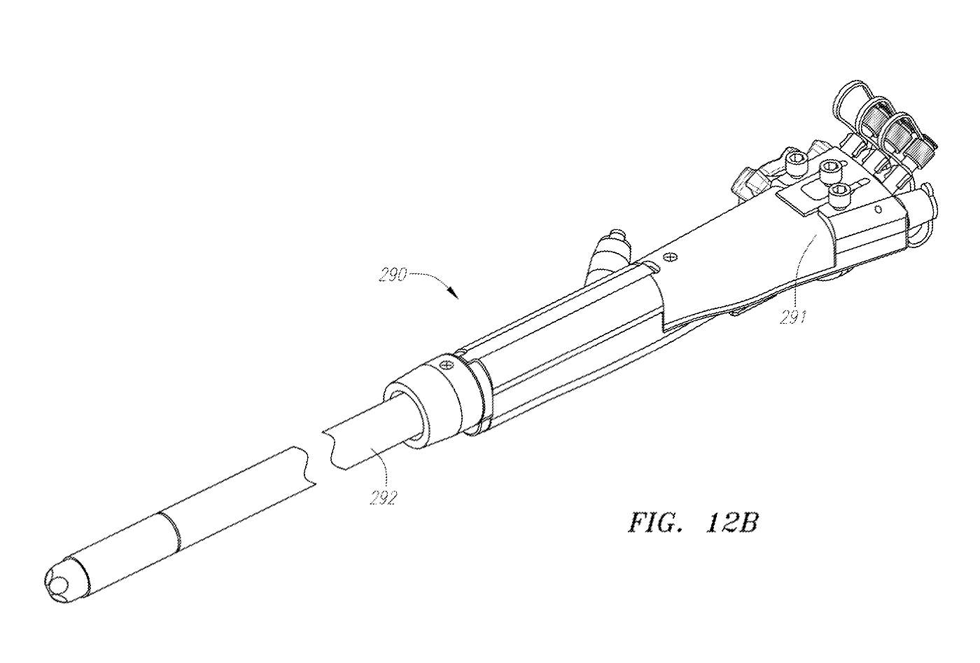

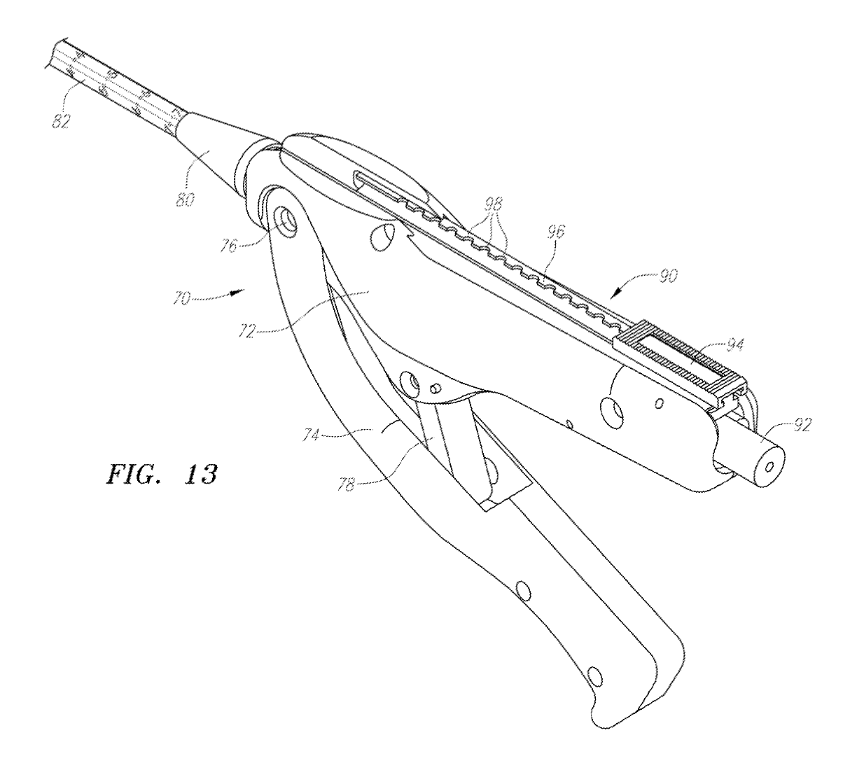

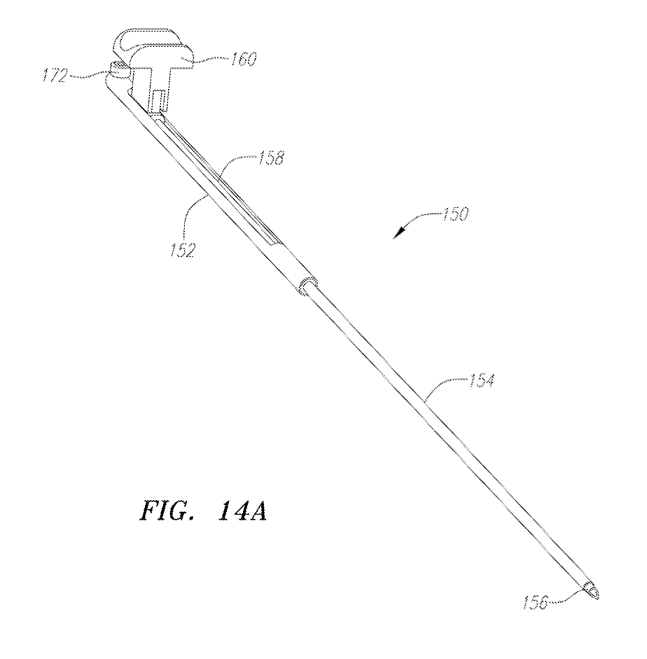

In several embodiments, the delivery device 210 and needle deployment assembly 260 are advanced into the gastrointestinal lumen using an endoscopic or endolumenal access system such as those described in the United States patent applications referenced above in Table 1. Examples of endoscopic and endolumenal access systems 290 are shown in FIGS. 12A and 12B. The endoscopic or endolumenal access systems 290 illustrated in FIGS. 12A and 12B each include a control mechanism 291 and a multi-lumen, steerable overtube 292 having several features that are described more fully in U.S. patent application Ser. Nos. 11/750,986 and 12/061,591, which were incorporated by reference above.

Turning to FIG. 13, an embodiment of an actuator mechanism 70 for a tissue anchor delivery device 208 is shown. The actuator mechanism 70 comprises an alternative embodiment to the handle 216 described above in relation to FIGS. 7 and 8. In the embodiment shown, the actuator mechanism 70 is configured to actuate both the tissue manipulation assembly 210 and the needle deployment assembly 260 independently of one another in order to grasp tissue and deploy a tissue anchor assembly 48 in separate steps. A brief description of the actuator mechanism 70 is provided below. Further detail is contained in U.S. patent application Ser. No. 12/486,578, which was incorporated by reference above.

The illustrated actuator mechanism 70 includes a main housing 72 and a handle body 74 that is pivotably attached to the main housing by a hinge pin 76, such that a user is able to grasp the main housing 72 and handle body 74 in one hand and actuate the mechanism by pulling the handle body 74 toward the main housing 72. A linkage arm 78 is interposed between the main housing 72 and the handle body 74, as discussed in more detail below. A nose cone 80 is attached to the distal end of the main housing 72 and surrounds the proximal end 82 of the tubular body 212.

In the embodiment shown, the proximal end 82 of the tubular body 212 is formed of a rigid material such as a rigid polymer material or stainless steel tubing. The remainder of the tubular body 212 is flexible and is formed of materials used to form the insertion portion of endoscopes and endoscopic devices. In alternative embodiments, the tubular body portion is formed of a composite tube that includes one or more polymeric materials (e.g., Pebax) and one or more braided layers (e.g., stainless steel or polymeric braid) to provide the tubular body 212 with improved torque transmission and resistance to stretching.

A needle deployment assembly actuation mechanism 90 includes a needle launch bushing 92, a needle launch button 94, and a needle launch track 96. The needle launch track 96 includes a plurality of substantially equally spaced, scallop-shaped cutouts 98 formed along the length of the track 96. More details of the structure and function of the needle deployment assembly actuation mechanism 90 is provided in the '578 application referenced above.

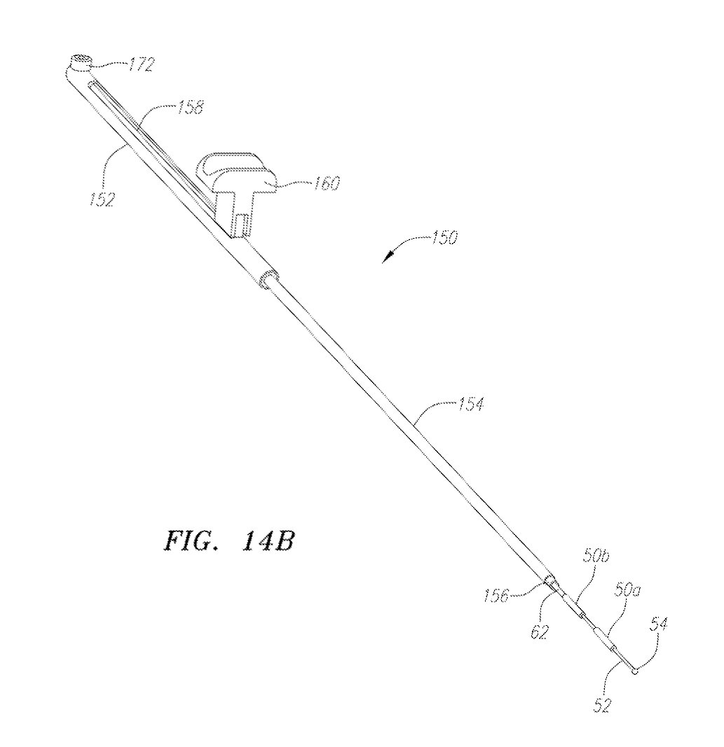

Turning next to FIGS. 14A and 14B, another embodiment of a needle deployment assembly 150 is shown. The needle deployment assembly 150 is configured for use with the actuator mechanism 70 described above in relation to FIG. 13. The assembly 150 includes a rigid body portion 152 and a flexible catheter portion 154 extending distally from the distal end of the rigid body portion 152. A needle 156 is fixed to the distal end of the catheter portion 154. The upper surface of the rigid body portion 152 includes an elongated groove 158 that provides access into the generally tubular rigid body 152. A suture deployment button 160 extends from the interior of the rigid body portion 152 through the groove 158. Additional details of the construction and operation of the needle deployment assembly 150 are contained in U.S. patent application Ser. No. 12/486,578, which was incorporated by reference above.

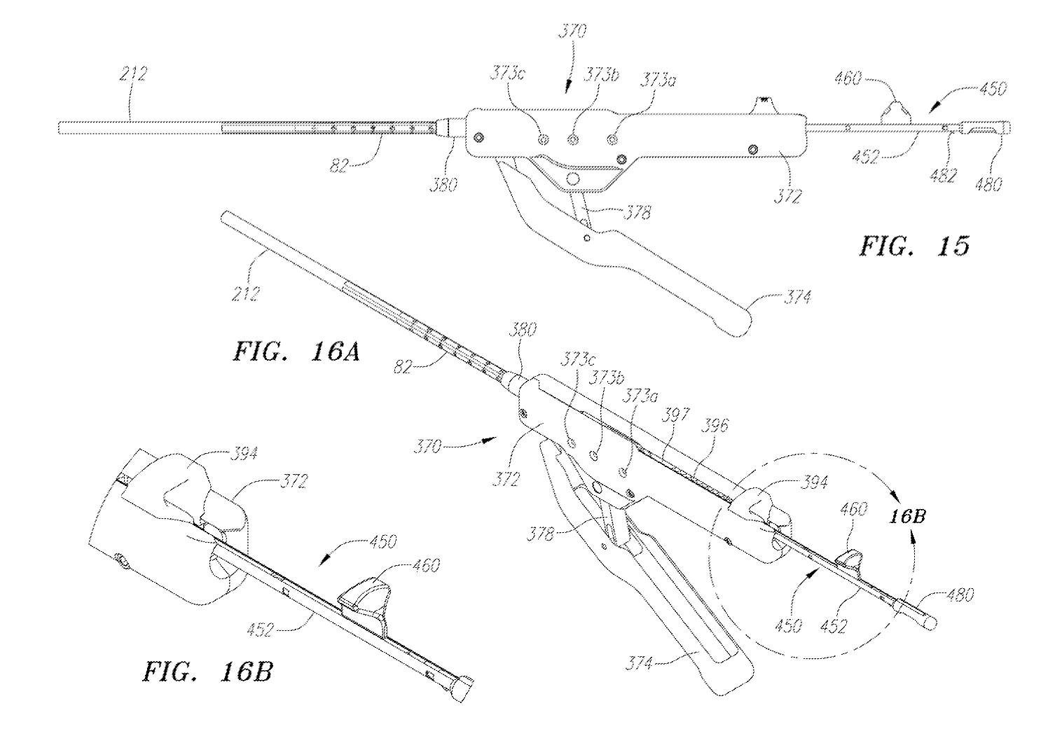

Turning to FIGS. 15 and 16A-B, another embodiment of an actuator mechanism 370 for a tissue anchor delivery device 208 is shown. The actuator mechanism 370 comprises another alternative embodiment to the handle 216 described above in relation to FIGS. 7 and 8 and the actuator mechanism 70 described above in relation to FIG. 13. In the embodiment shown, the actuator mechanism 370 is configured to actuate both the tissue manipulation assembly 210 and the needle deployment assembly 260 independently of one another in order to grasp tissue and deploy a tissue anchor assembly 48 in separate steps. The actuator mechanism 370 also includes a number of additional features that will be described in relation to the Figures.

Turning first to FIGS. 15 and 16A-B, the illustrated actuator mechanism 370 includes a main housing 372 and a handle body 374 that is pivotably attached to the main housing by a hinge pin 376, such that a user is able to grasp the main housing 372 and handle body 374 in one hand and actuate the mechanism by pulling the handle body 374 toward the main housing 372. A linkage arm 378 is interposed between the main housing 372 and the handle body 374, as discussed in more detail below. A nose cone 380 is attached to the distal end of the main housing 372 and surrounds the proximal end 82 of the tubular body 212.

A needle deployment assembly actuation mechanism 390 includes a needle launch button 394, and a needle launch track 396. The needle launch track 396 includes a plurality of substantially equally spaced, scallop-shaped cutouts 398 formed along the length of the track 396. More details of the structure and function of the needle deployment assembly actuation mechanism 390 are contained in U.S. patent application Ser. No. 12/486,578, which was incorporated by reference above.

As shown in FIGS. 15 and 16A, the exterior surface of the main housing 372 includes a plurality of handle position indicator windows, including a “fully open window” 373a, a “neutral position window” 373b, and a “fully closed window” 373c. Each of the windows comprises an opening into the interior of the main housing 372 that is located within the travel path of the drive arm 378b of the linkage arm. Accordingly, as the actuator mechanism 370 is transitioned between the fully open, neutral, and fully closed positions, the drive arm 378b is located within the corresponding window, thereby providing a visual indicator on the main housing 372 of the position of the actuator mechanism 370. The position indicator may be highlighted by providing a lighted, brightly colored, or otherwise visually observable member on the drive arm 378b in a position observable through the windows 373a, 373b, 373c.

Additional details concerning the structure and function of the devices and methods described above in relation to FIGS. 13 through 20A-B are contained in U.S. patent application Ser. No. 12/486,578, filed Jun. 17, 2009, which is hereby incorporated by reference in its entirety.

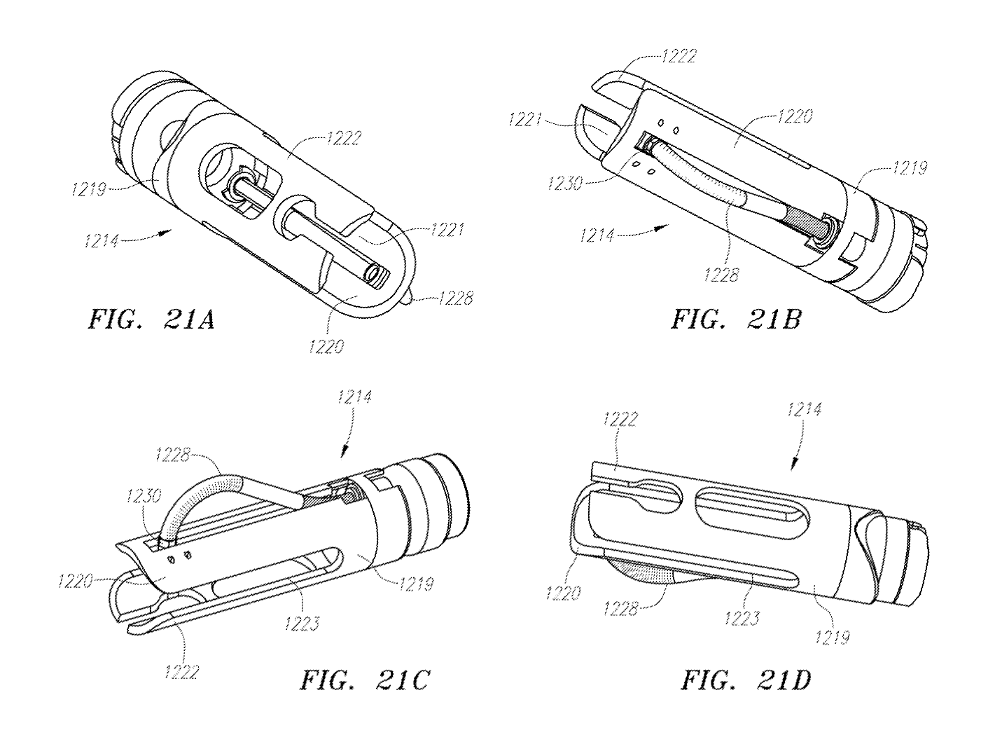

Turning next to the device embodiments shown in FIGS. 21A-D through 26, there are shown several embodiments of a tissue anchor delivery device 1108 having a distal end effector that is configured to be advanced endoscopically or endolumenally, e.g., transorally, transanally, transvaginally, transgstrically, etc., into the patient's body, e.g., the stomach. Using the tissue anchor delivery device 1208, tissue may be engaged and tissue anchors deployed through tissue folds, thereby providing a surgeon or other practitioner with the capability of reconfiguring tissue from outside the patient's body.

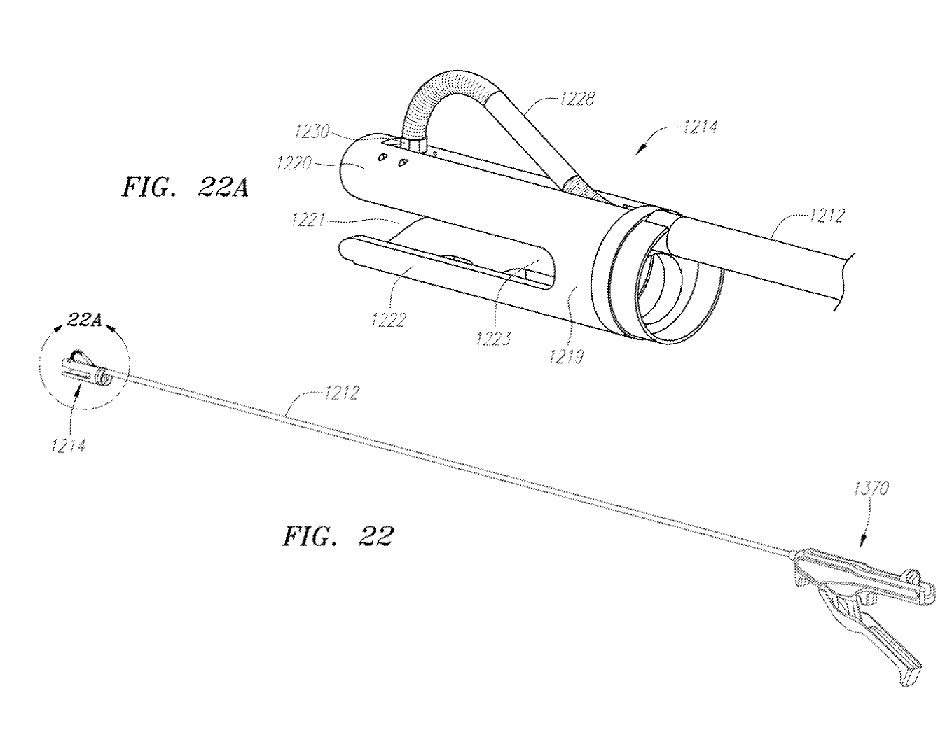

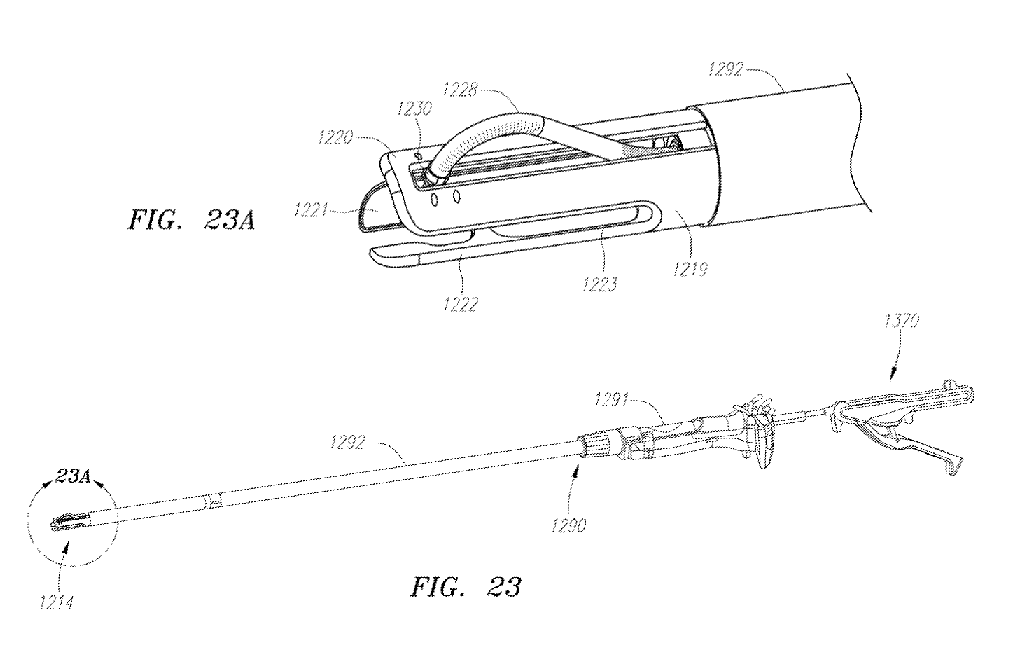

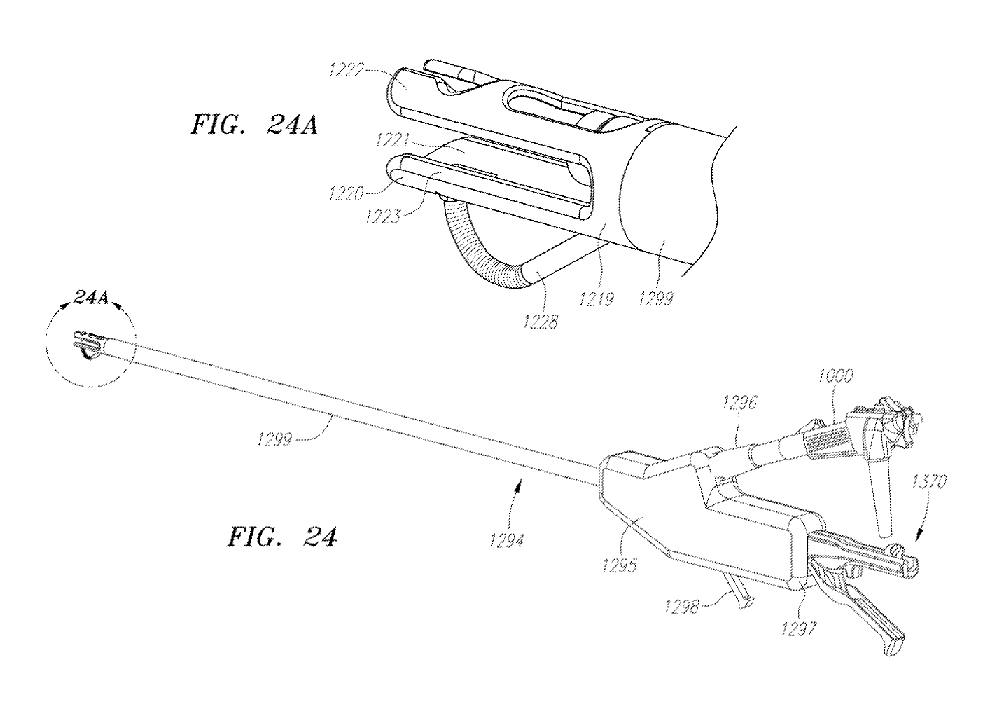

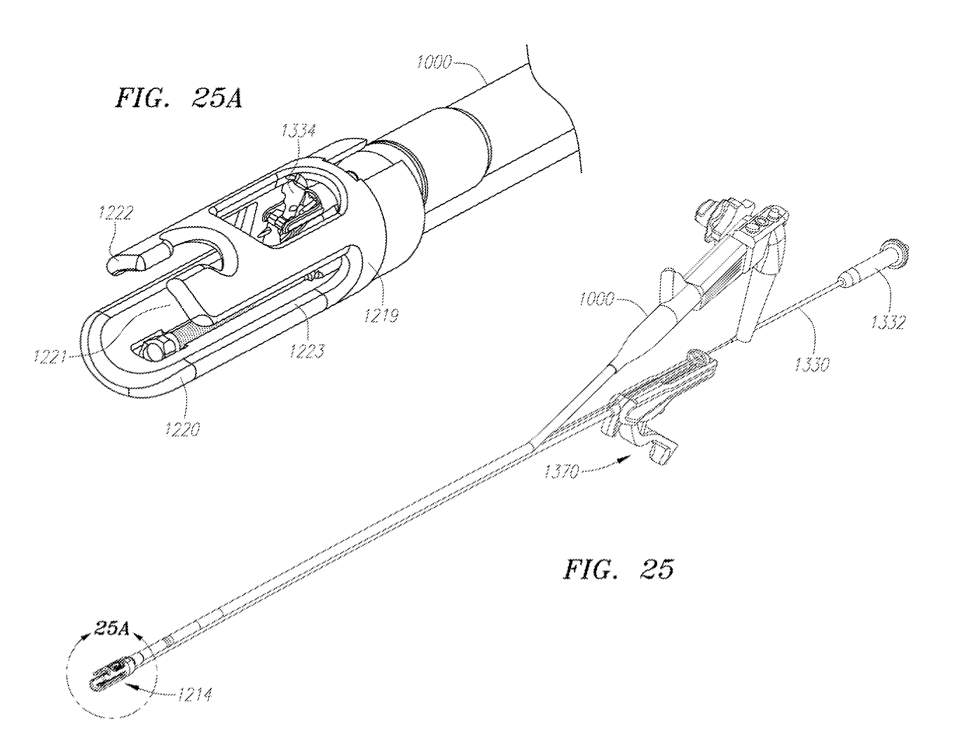

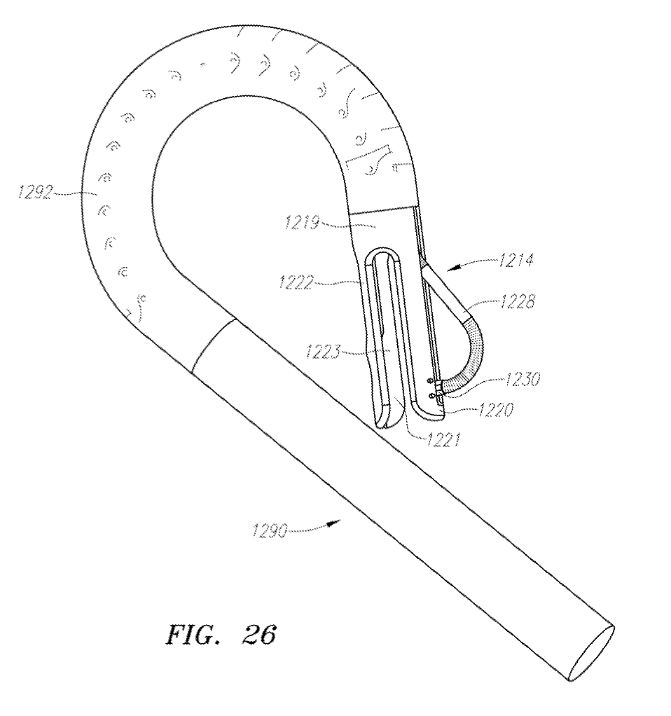

The tissue anchor delivery device 1208 shown in FIGS. 21A-D through 26 includes generally comprises a distal end effector 1214 attached to the distal end of an elongated shaft and under the control of an actuator. In some embodiments, such as shown in FIGS. 22 and 22A, the elongated shaft comprises a tubular body 1212 having a construction like that of the tubular body 212 described above in relation to FIGS. 7 through 20A-B. In other embodiments, such as shown in FIGS. 25 and 25A, the elongated shaft comprises an endoscope 1000 having conventional construction and features known to those skilled in the art. In still other embodiments, such as shown in FIGS. 23 and 23A and 26, the elongated shaft comprises an endoscopic access device 1290 having a construction and features like those of the endoscopic access devices 290 described above in relation to FIGS. 12A-B. In still other embodiments, such as shown in FIGS. 24 and 24A, the elongated shaft comprises a steerable sheath 1294, described in more detail below.

The actuator component of the delivery device 1208 embodiments shown in FIGS. 21A-D through 26 generally comprises an actuator mechanism 1370 having a construction and features like those of the actuator mechanism 370 described above in relation to FIGS. 15 and 16A-B.

In the embodiments shown, the end effector 1214 includes a generally cylindrical base portion 1219, an upper extension 1220 extending generally distally away from the base portion 1219, and a lower extension 1222 also extending generally distally away from the base portion in a generally parallel relation with the upper extension 1220. The upper extension 1220 and lower extension 1222 thereby together define: (1) a tissue acquisition and folding space 1221 between the upper extension 1220 and lower extension 1222, and (2) a slot 1223 on each side of the end effector 1214 in communication with the tissue acquisition and folding space 1221.

A launch tube 1228 is attached to the end effector 1214 via a launch tube pivot 1230. The launch tube 1228 and launch tube pivot 1230 have a construction and features comparable to those of the launch tube 228 and pivot 230 for the embodiments described above in relation to FIGS. 7 through 20A-B. The launch tube 1228 is actuated by the actuator mechanism 1370 via a drive tube that causes the launch tube 1228 to selectively transform to the arcuate shape shown, for example, in FIGS. 21C and 22 and 22A. Once the launch tube 1228 is placed in the arcuate shape, the tissue anchor delivery device 1208 is prepared to deploy a needle deployment assembly 450, through the launch tube 1228, by way of the actuator mechanism 1370, in the manner described above.

Turning to FIGS. 23 and 23A and 26, in the embodiment shown, the endoscopic access device 1290 includes a control mechanism 1291 and a multi-lumen, steerable overtube 1292 having several features that are described more fully in U.S. patent application Ser. Nos. 11/750,986 and 12/061,591, which were incorporated by reference above. The actuator mechanism 1370 of the tissue anchor delivery device 1208 is held in a fixed relationship relative to the control mechanism 1291 of the access device 1290. For example, a tool lock may be provided on the control mechanism 1291 and may be locked upon the exterior surface of the tubular body 1212 of the delivery device 1208. In other embodiments, the end effector 1214 is fixedly attached to the distal end of the steerable overtube 1292, (e.g., see FIG. 23A). The steering capability of the endoscopic access device 1290 provides the user with the ability to steer the end effector 1214 into locations within the body of a patient that are difficult to reach, such as portions of the stomach that require a retroflex approach. (See, e.g., FIG. 26).

Turning to FIGS. 24 and 24A, in the embodiment shown, the steerable sheath 1294 includes a handle portion 1295 having an endoscope port 1296 through which the endoscope 1000 extends, and a delivery device port 1297 through which the anchor delivery device 1208 extends. A steering control 1298 is also provided on the handle portion 1295. The steering control 1298 controls steering of the steerable shaft 1299 via one or more steering wires (not shown). In some embodiments, steering of the steerable shaft 1299 is provided in only a single direction, such as by a single steering wire that is tensioned by the steering control 1298. In other embodiments, steering of the steerable shaft 1299 is provided in two directions. In still other embodiments, full four-way steering of the steerable shaft 1299 is provided.

In the embodiments shown in FIGS. 25 and 25A, the end effector 1214 of a tissue anchor delivery device 1208 is attached to the distal end of an endoscope 1000, and the tubular body 1212 of the delivery device 1208 is attached to the exterior surface of the endoscope 1000, such as with an adhesive, or with connectors, or with other mechanisms or methods described elsewhere herein. In addition, a tissue grasper 1330 extends through the working channel 1002 of the endoscope. The tissue grasper 1330 includes a handle portion 1332 extending proximally of the actuator mechanism 1370 of the delivery device 1208, and a pair of jaws 1334 that extend into the tissue acquisition space 1221 between the upper extension 1220 and lower extension 1222 of the end effector 1214. Additional details concerning the structure and function of the tissue grasper 1330 are contained in U.S. patent application Ser. No. 11/736,539, filed Apr. 17, 2007, which is hereby incorporated by reference in its entirety The tissue grasper 1330 is translatable and rotatable within the working channel 1002 of the endoscope, thereby providing the user with the ability to extend the tissue grasper 1330 outside of the tissue acquisition space 1221 to acquire tissue, then pull the tissue back into the tissue acquisition space 1221 for reconfiguration.

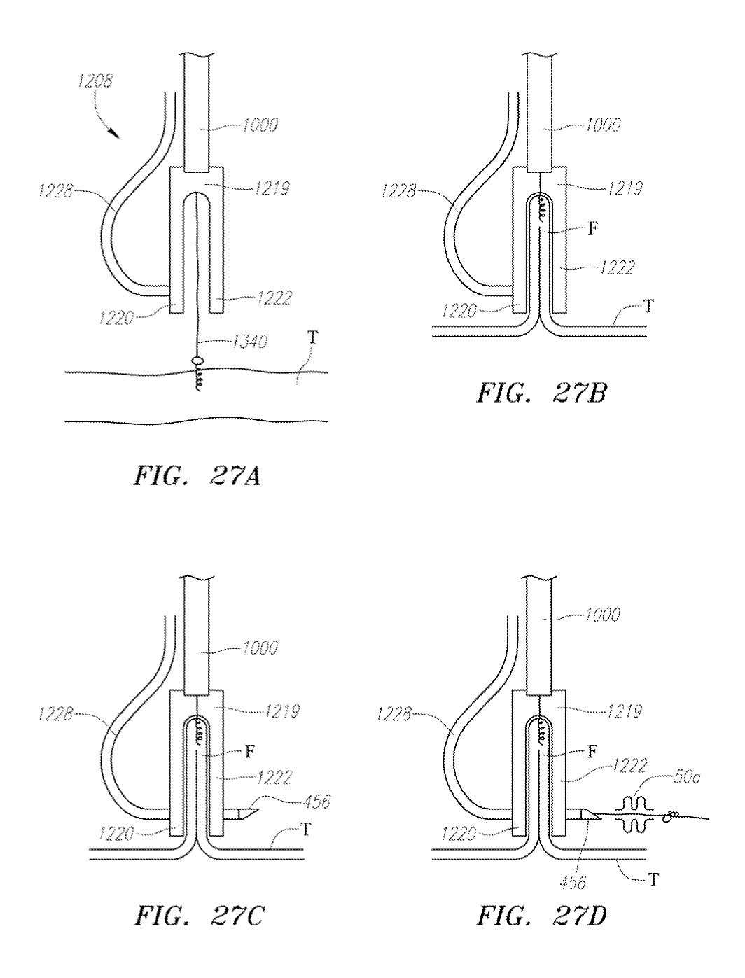

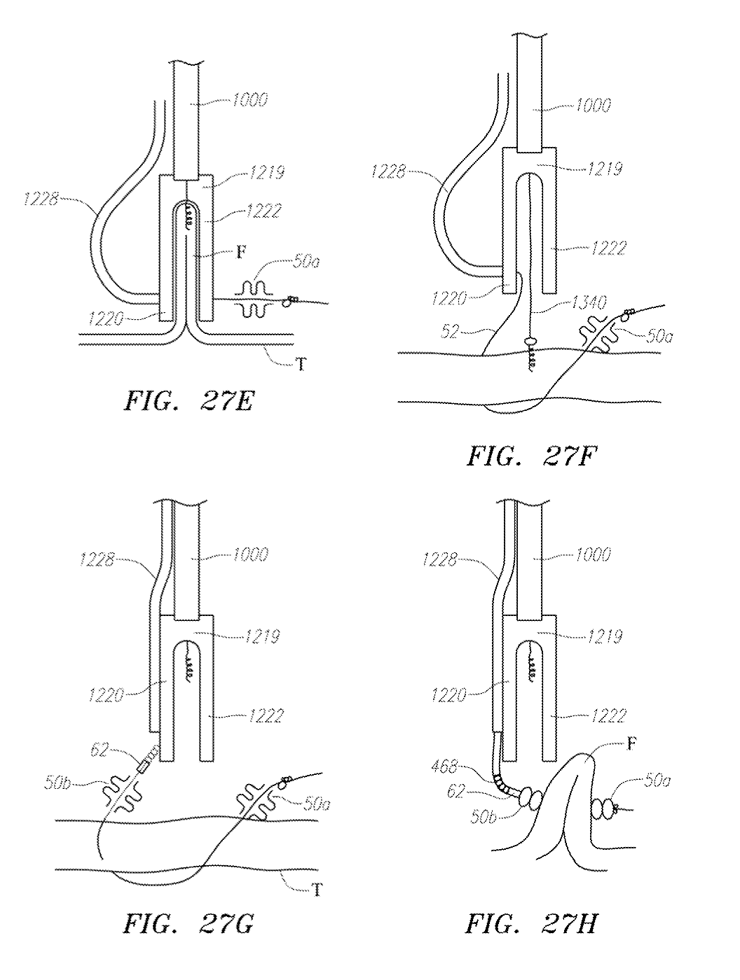

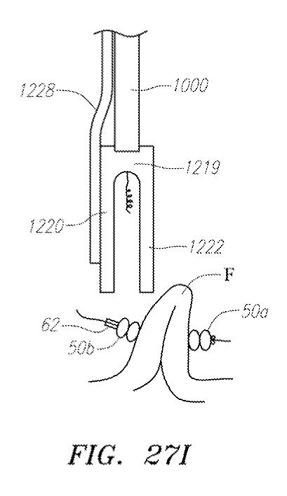

Turning next to FIGS. 27A-I, a method of deploying a tissue anchor using an embodiment of the tissue anchor delivery device 1208 is shown. In the embodiment shown, the delivery device 1208 is similar to that described above in relation to FIGS. 25 and 25A, comprising a distal end effector 1214 attached to the distal end of an endoscope 1000. However, rather than the tissue grasper 1330, the embodiment shown in FIGS. 27A-I includes a tissue retractor 1340 having a helical coil attached at a distal end of an elongated shaft. In a first step, shown in FIG. 27A, the distal end effector 1214 is brought into proximity of the target tissue T, and the tissue retractor 1340 is extended toward the tissue. Once in contact with the tissue, the helical coil of the tissue retractor 1340 is rotated so that it embeds into the tissue T. Next, as shown in FIG. 27B, the retractor 1340 is retracted within the working channel 1002 of the endoscope 1000, thereby pulling the tissue T into the tissue acquisition and folding space 1221 between the upper extension 1220 and lower extension 1222 of the end effector 1214. This action forms a tissue fold F in the tissue. At this point, if the launch tube 1228 has not yet been activated, it is then activated by way of the actuator mechanism 1370 (not shown in FIGS. 27A-I). Once the launch tube 1228 is actuated, a needle deployment assembly 450 is deployed, causing the needle 456 to extend through the tissue fold F formed within the tissue acquisition and folding space 1221. (See FIG. 27C). After the needle 456 has traversed the tissue fold F, the distal anchor 50a is expelled from the distal end of the needle 456 on the lower extension 1222 side of the tissue fold F, as shown in FIG. 27D. Next, the needle deployment assembly 450 is retracted from the tissue fold F, and into the lumen of the launch tube 1228. (See FIG. 27E). The helical coil retractor 1340 is then extended, releasing the tissue fold F. (See FIG. 27F). At this point, the distal anchor 50a is retained in the target tissue T, and the suture 52 extends through the tissue T, but the tissue fold F may flatten out or disappear. Next, the launch tube 1228 is deactivated, and the needle deployment assembly pusher 468 is extended to deploy the proximal anchor 50b and cinch 62 from the needle deployment assembly 450, which is still located in the lumen of the launch tube 1228. (See FIG. 27G). The pusher 468 is extended further until the tissue anchor assembly 48 is fully deployed by the cinch 62, as shown in FIG. 27H. Finally, the suture 52 extending between the anchor assembly 48 and the deployment device 1208 is severed or broken away, (see FIG. 27I), and the deployment device is withdrawn, leaving a tissue fold F retained by a tissue anchor assembly 48.

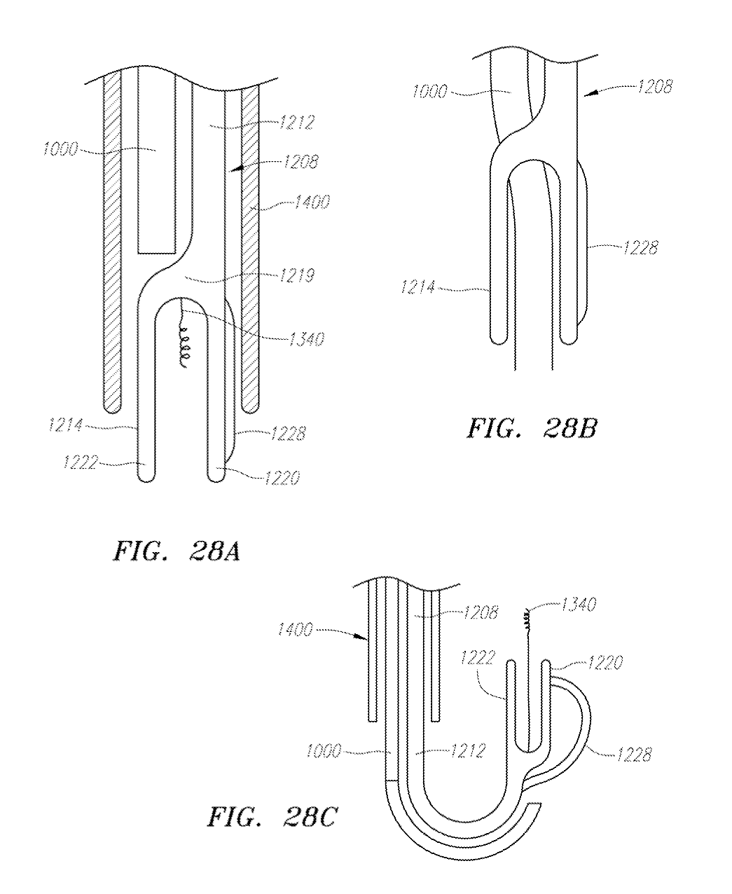

FIGS. 28A-C show the distal region of a tissue anchor deployment device 1208 having a construction similar to that described above in relation to FIGS. 22 and 22A. In the embodiment shown in FIG. 28A, the deployment device 1208 is disposed within the lumen of an overtube 1400 next to an endoscope 1000. The distal end of the endoscope 1000 rests against the proximal side of the base portion 1219 of the distal end effector 1214 of the delivery device 1208 while the devices are disposed within the lumen of the overtube 1400. In an alternative embodiment shown in FIG. 28B, the distal portion of the endoscope 1000 is able to be routed through a gap or passage formed in the base portion 1219.

The deployment device 1208 embodiment shown in FIGS. 28A-C includes an independent steering capability. For example, in the embodiment, the actuator mechanism 1370 (not shown in FIGS. 28A-C) is provided with steering controls that are actuatable to provided tension to one or more steering wires that extend through the tubular body 1212, thereby providing the user with the capability of steering the distal region of the device (including the distal end effector 1214). FIG. 28C shows the deployment device 1208 and endoscope 1000 extended through the lumen of the overtube 1400. The steering control on the actuator mechanism 1370 (not shown) is actuated to steer the tubular body 1212 into the retroflexed position shown in the Figure. In this way, the distal end effector 1214 of the device is able to be controlled and moved by the user to a target location within a patient's body independently, i.e., without relying upon the steering capability of a separate instrument, such as an endoscope 1000 or other access device.

The devices described herein are suitable for use in many diagnostic and therapeutic procedures in which tissue manipulation and securement is performed endoscopically or endolumenally. Examples of such procedures include endolumenal treatment of obesity (see, e.g., U.S. patent application Ser. No. 12/409,335, filed Mar. 23, 2009, and U.S. Provisional Patent Application Ser. No. 61/239,709, filed Sep. 3, 2009, each of which is hereby incorporated by reference), revision of obesity procedures (see, e.g., U.S. patent application Ser. No. 11/342,288, filed Jan. 27, 2006, hereby incorporated by reference), treatment of gastroesophageal reflux disease (GERD) (see, e.g., U.S. patent application Ser. No. 11/290,304, filed Nov. 29, 2005, hereby incorporated by reference), gastrotomy closure procedures (see, e.g., U.S. patent application Ser. No. 11/238,279, filed Sep. 28, 2005, hereby incorporated by reference), wound closure, fistula repair, and other procedures in which two or more portions of tissue are grasped, manipulated, approximated, or secured. Additional examples of procedures are described in the other patent applications incorporated by reference herein.

Although various illustrative embodiments are described above, it will be evident to one skilled in the art that various changes and modifications are within the scope of the invention. It is intended in the appended claims to cover all such changes and modifications that fall within the true spirit and scope of the invention.

What is claimed is:

1. Apparatus for endoscopically manipulating tissue comprising:
an overtube having at least one internal lumen;
an endoscope extending through the at least one overtube lumen and being translatable and rotatable relative to the overtube;
a tissue plication device extending through the at least one overtube lumen adjacent to the endoscope, the tissue plication device comprising:
an elongated body,
an end effector disposed at a distal region of the main body, the end effector including a first extension and a second extension defining a space therebetween,
a tissue grasping device extendable into the space defined between the upper extension and lower extension, and
a launch tube having a flexible distal region attached to the first extension at or near a distal end thereof;
wherein the tissue plication device includes a steering controller that is actuatable to steer a distal region of the tissue plication device.

2. The apparatus of claim 1, further comprising a plurality of steering wires extending through the tissue plication device and operatively connected to the steering control.

3. The apparatus of claim 1, wherein the tissue plication device includes a handle at or near a proximal end thereof, with the steering controller being disposed on the handle.

4. The apparatus of claim 1, further comprising a needle deployment assembly having a flexible sheath and a tissue piercing tip that is translatable through the launch tube.

5. The apparatus of claim 5, further comprising an anchor assembly disposed within the needle deployment assembly.

6. The apparatus of claim 1, wherein the flexible distal region of the launch tube has a first position that is substantially aligned with the first extension and a second position in which the flexible distal region has a generally arcuate shape.
